(19) United States
(12) Patent Application Publication
(10) Pub. No.: US 2025/0359559 A1
(43) Pub. Date: Nov. 27, 2025

(54) Apparatus for facilitating ultrasound scanning

(71) Applicant: MEQ PROBE PTY LTD, Victoria (AU)

(72) Inventors: Remo CARBONE, Victoria (AU); Jordan YEOMANS, Victoria (AU); Thomas PEMBERTON, Victoria (AU)

(21) Appl. No.: 18/873,232

(22) PCT Filed: Jun. 9, 2023
(86) PCT No.: PCT/AU2023/050512

§ 371 Date: Dec. 9, 2024

(30) Foreign Application Priority Data
Jun. 9, 2022 (AU) 2022901585

(51) Int. Cl. A22B 5/00; A61B 8/00

(52) U.S. Cl. CPC A22B 5/007; A61B 8/4209; A61B 8/4272; A61B 8/4461; A61B 8/546

(57) ABSTRACT

An apparatus for facilitating ultrasound scanning of objects. The apparatus comprises a housing defining a reservoir. The housing comprises: a body comprising a transducer mounting, the transducer mounting configured to support an ultrasound transducer; a flange extending from the body to define side walls of the reservoir; and a first flexible membrane mounted on a free end of the flange, the first membrane defining an external contact surface for making contact with an object to be scanned, and the first flexible membrane configured to allow ultrasound waves to propagate therethrough. The reservoir is configured to retain ultrasound propagation fluid to facilitate ultrasound wave propagation between the transducer and the first flexible membrane.

### TECHNICAL FIELD

Described embodiments relate to apparatus for facilitating ultrasound scanning. Some embodiments in particular relate apparatus for facilitating ultrasound scanning of non-uniform shaped objects, such as live animals.

### BACKGROUND

In the meat industry, grading of meat on carcasses is typically performed after an animal has been slaughtered and processed.

Existing techniques used to assess carcass attribute while animals are alive relies on an expert placing the ultrasound device in a precise location to properly assess the carcass attribute in question. Specific training is required for operators to be able to reliably produce ultrasound images of high quality and consistency.

Current devices for us on live animals use a stand-off material with a predefined curvature built in, the predefined curvature and the inability of the material to adjust its curvature significantly limits its application to target specimens that very closely match the shape of the inbuilt curve.

Accordingly, existing solutions are limited in their applications due to the physical shape of the scanning devices, and the expertise required to operate the scanning devices. This results in ultrasound scanning devices that are not useable across a wide range of targets or locations, without expert knowledge.

It is desired to address or ameliorate one or more shortcomings or disadvantages associated with such prior art, or to at least provide a useful alternative hereto.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

Some embodiments relate to an apparatus for facilitating ultrasound scanning of objects, the apparatus comprising: a housing defining a reservoir, the housing comprising: a body comprising a transducer mounting, the transducer mounting configured to support an ultrasound transducer; a flange extending from the body to define side walls of the reservoir; and a first flexible membrane mounted on a free end of the flange, the first membrane defining an external contact surface for making contact with an object to be scanned, and the first flexible membrane configured to allow ultrasound waves to propagate therethrough; wherein the reservoir is configured to retain ultrasound propagation fluid to facilitate ultrasound wave propagation between the transducer and the first flexible membrane.

The transducer mounting may be disposed within the reservoir of the housing. The housing may comprise a second membrane opposite to the first flexible membrane, and wherein the transducer mounting is disposed on an external surface of the housing, such that ultrasound waves emitted from the transducer are configured to propagate through the second membrane into the reservoir, through ultrasound propagation fluid in the reservoir and through the first flexible membrane. The first flexible membrane may comprise an elastomeric material. The membrane may comprise a sheet of silicone having a thickness of about 0.1 mm to 3 mm. The free end of the flange may comprise a substantially curved surface such that the first flexible member mounted thereon provides a substantially curved external contact surface.

The flange may comprise, or be composed of, moulded silicone. The flange and/or the first flexible membrane may be removably coupled to the housing.

In some embodiments the apparatus may further comprise a track disposed along an inner wall of the housing, the track being configured to cooperate with the transducer mounting to allow the transducer mounting to be selectively moved along the track and assume a range of positions within the reservoir. The track may be configured to allow the mounting to move in a first direction and to move in a second direction, wherein the second direction is orthogonal to the first direction. The transducer mounting may comprise at least one shaft, and wherein the apparatus may further comprise: at least one motor, external to the reservoir; at least one magnetic motor coupling connected to the motor and configured to magnetically engage at least one magnetic shaft coupling attached to the at least one shaft. The at least one motor may comprise at least one stepper motor. The at least one motor may comprise at least one servomotor.

The housing may comprise at least one handle extending from the housing to allow a user to hold the apparatus during operation.

The reservoir may be filled with ultrasound propagation fluid, and wherein the ultrasound propagation fluid is configured to propagate ultrasounds waves between the transducer and the first membrane.

The apparatus may further comprise an ultrasound transducer mounted on the transducer mounting. The apparatus may further comprise a pump provided within the housing, configured to mix ultrasound propagation fluid stored within the reservoir. The pump may further comprise a shaft connected to at least one magnetic shaft coupling and a nut, wherein the at least one magnetic shaft coupling is configured to magnetically couple to a magnetic motor coupling arranged to be drive by a motor, and responsive to rotation of the magnetic motor coupling, the magnetic shaft coupling rotates, thereby causing the shaft to rotate and the nut to move back and forth along the shaft providing a piston pump action. The apparatus may comprise at least one heating element, provided within the housing, and configured to selectively heat ultrasound propagation fluid stored within the reservoir. The apparatus may comprise at least one temperature sensor, within the housing, configured to determine a temperature of ultrasound propagation fluid stored within the reservoir.

The apparatus may further comprise at least one heating element, provided within the housing, and configured to selectively heat ultrasound propagation fluid stored within the reservoir; at least one temperature sensor, within the housing, configured to determine a temperature of ultrasound propagation fluid stored within the reservoir; and a controller having a memory and one or more processors, wherein the controller is configured to: receive temperature data from the at least one temperature sensor, the temperature data being indicative of the temperature of the propagation fluid within the reservoir; responsive to the temperature of the propagation fluid being less than a first threshold level, activate the at least one heating element to thereby heat the propagation fluid; and responsive to the temperature of the propagation fluid being greater than a second threshold level, deactivate the at least one heating element.

Some embodiments relate to an apparatus comprising: a body; an ultrasound transducer connected to the body; a flange extending from the body; and a flexible membrane mounted on the flange, wherein the flexible membrane, flange and body cooperate to define a reservoir between the ultrasound transducer and the flexible membrane, the reservoir being at least partially surrounded by the flange and configured to contain ultrasound propagation fluid between the ultrasound transducer and the flexible membrane.

Some embodiments relate to a system comprising the apparatus for facilitating ultrasound scanning of objects, wherein the apparatus further comprises a winch mounting, and a winch configured to couple to the winch mounting and wherein the one or more processors are configured to execute the instructions to operate the winch. The winch may comprise a motorized winch. The system may further comprise a controller having a memory and one or more processors, wherein the memory comprises instructions for operating the winch according to one or more predefined settings. The controller may comprise a user interface for receiving one or more values for the respective one or more predefined settings, and optionally operational zone ranges for the winch mounting. Some embodiments relate to a system comprising a first storage tank, configured to store a quantity of water; a second storage tank, configured to store a quantity of ultrasound propagation fluid; at least one heating element, provided within the first storage tank, and configured to selectively heat water stored within the first storage tank; a conduit arranged to extend from a first fluid port in the first storage tank through the second storage tank and to a second fluid port in the first storage tank; a pump, configured to convey water from the first storage tank through the conduit; at least one temperature sensor, within the second storage tank, configured to determine a temperature of the ultrasound propagation fluid stored within the second storage tank; and a controller, wherein the controller is configured to receive temperature data from the at least one temperature sensor, the temperature data being indicative of the temperature of the propagation fluid within the second storage tank; operate the heating element to thereby heat water in the first storage tank; responsive to the temperature of the propagation fluid within the second storage tank being less than a first threshold level, activate the pump to circulate the heated water from the first storage tank into the conduit in the second storage tank, thereby heating propagation fluid within the second storage tank; and responsive to the temperature of the propagation fluid being greater than a second threshold level, deactivate the pump.

The system may further comprise a propagation fluid pump provided within the propagation fluid tank and configured to circulate the propagation fluid within the propagation fluid tank. The system may further comprise a dispenser configured to dispense ultrasound propagation fluid from the second storage tank.

### BRIEF DESCRIPTION OF DRAWINGS

Various ones of the appended drawings merely illustrate example embodiments of the present disclosure and cannot be considered as limiting its scope.

FIG. 1 is schematic of a process for performing an ultrasound on an object, such as a live animal, according to some embodiments;

FIG. 2A is a top perspective view of an apparatus for facilitating ultrasound scanning, according to some embodiments;

FIG. 2B is a perspective bottom view of the apparatus of FIG. 2A with a flexible membrane of the apparatus removed, according to some embodiments;

FIG. 2C is a side view of the apparatus of FIG. 2A, according to some embodiments;

FIG. 2D is a bottom view of the apparatus of FIG. 2A, according to some embodiments;

FIG. 3A is a perspective view of a flange and flexible membrane of the apparatus of FIG. 2A;

FIG. 3B is a cut away view of the flange and flexible membrane of FIG. 3A;

FIG. 4 is a first top perspective exploded view of the apparatus of FIG. 2A, according to some embodiments;

FIG. 5 is a schematic of a scanning system comprising the apparatus of FIG. 2A connected to a safety mechanism via a winch mount, according to some embodiments;

FIG. 6A is a schematic side view of a cattle enclosure comprising a frame with the scanning system of FIG. 4 mounted thereto, according to some embodiments;

FIG. 6B is a schematic perspective view of the cattle enclosure of FIG. 5, according to some embodiments;

FIG. 7A is an example ultrasound image captured using a prior art apparatus for facilitating ultrasound scanning;

FIG. 7B is an example ultrasound image captured by the apparatus of FIG. 2A, according to the described embodiments;

FIG. 8A is a perspective view of a motor and shaft coupling of the apparatus of FIG. 2A, according to some embodiments;

FIG. 8B is a sectional perspective view of the motor and shaft coupling of the apparatus of FIG. 2A, according to some embodiments;

FIG. 8C is a sectional perspective view of the motor and shaft coupling in connection with a motor system of the apparatus of FIG. 2A, according to some embodiments;

FIG. 9A is a bottom perspective view of the apparatus of FIG. 2A, with flexible membrane visible, according to some embodiments;

FIG. 9B is perspective view of a cavity of the apparatus of FIG. 2A, according to some embodiments;

FIG. 10A is a top perspective view of an apparatus for facilitating ultrasound scanning, according to some embodiments;

FIG. 10B is a top perspective view of the apparatus of FIG. 10A, with some components removed, according to some embodiments;

FIG. 11A is a top perspective view of the apparatus of FIG. 10A, showing a partial internal view of the apparatus, according to some embodiments;

FIG. 11B is a top view of the apparatus of FIG. 10A, showing a partial internal view of the apparatus, according to some embodiments;

FIG. 12A is a first side sectional view of the apparatus of FIG. 10A, according to some embodiments;

FIG. 12B is a second side sectional view of the apparatus of FIG. 10A, according to some embodiments;

FIG. 13A is a first perspective view of a pump, according to some embodiments;

FIG. 13B is a perspective sectional view of the pump of FIG. 13A, according to some embodiments;

FIG. 13C is a second perspective view of a pump, according to some embodiments;

FIG. 13D is a further perspective view of the apparatus of FIG. 10A, showing a partial internal view of the apparatus, according to some embodiments;

FIG. 14A is a front sectional view of the pump of FIG. 13A, according to some embodiments;

FIG. 14B is a front sectional view of the apparatus of FIG. 10A, according to some embodiments;

FIG. 15 is a top sectional view of the apparatus of FIG. 10A, according to some embodiments;

FIG. 16 is an example ultrasound image captured by the apparatus of FIG. 10A, according to some embodiments;

FIG. 17A is a perspective view of an applied oil heater, according to some embodiments;

FIG. 17B is a perspective internal view of an applied oil heater, according to some embodiments; and

FIG. 17C is a partially diagrammatic front view of an applied oil heater, according to some embodiments.

### DETAILED DESCRIPTION

Described embodiments relate to apparatus for facilitating ultrasound scanning. Some embodiments in particular relate apparatus for facilitating ultrasound scanning of non-uniform shaped objects, such as live animals.

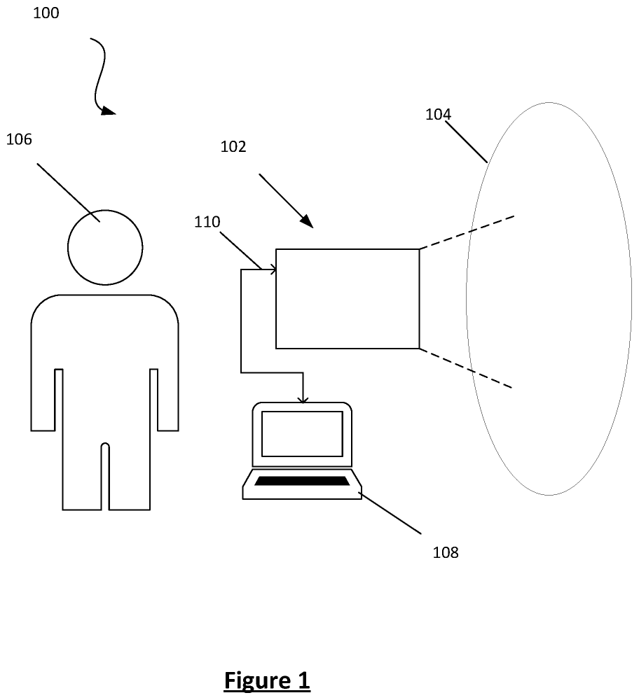

FIG. 1 depicts a schematic depicting a process for performing an ultrasound on an object, such as a live animal, using an apparatus 102, according to the described embodiments.

Before using the apparatus 102, object 104 to be scanned may need to be prepared. This may involve the application of a propagation medium such as oil or water to the object. In some embodiments, this may involve the removal of a fat layer in the case of brisket detection, or shaving hair from animals for veterinary use.

An external contact surface of the apparatus is positioned on an area of the object to be scanned. The ultrasound transducer is activated and ultrasound measurements of the object are taken.

The ultrasound measurements are transmitted from the transducer to a client device 108 by means of cable 110, for processing and assessment by user 106. Cable 110 may comprise a data cable, to ensure accurate and high quality data transmission. Cable 110 may further comprise a power cable to supply power to apparatus 102. In some embodiments, a wireless data connection may be used instead.

The client device 108 may comprise an application for processing the measurements and making determinations about the scanned object. For example, where the object is a live animal, the application may be configured to determine or classify a grade of meat of the object, which may depend on deduced intramuscular fat and/or muscle segmentation.

The quality of data acquired by the transducer and provided to the application makes a significant impact on the effectiveness of the application and any determinations being made thereby. The described apparatus may facilitate the acquisition of improved ultrasound scans.

According to some embodiments, the apparatus comprises a housing defining a reservoir configured to retain ultrasound propagating fluid, and a transducer mount arranged to support an ultrasound transducer. The housing comprises a body, and flange extending from the body and defining side walls of the reservoir, and a flexible membrane mounted on the free end of the flange, thereby enclosing the reservoir. The flexible membrane defines an external contact surface for making contact with an object to be scanned using the ultrasound transducer, such as the back of an animal. The flexible membrane is configured to allow ultrasound waves to propagate therethrough.

An ultrasound transducer is mounted to the transducer mounting and the reservoir is filled with ultrasound propagating fluid in a manner that mitigates any air gaps within the reservoir. In use, the external contact surface of the flexible membrane of the housing is positioned on an object to be scanned. Ultrasound signals emitted from the ultrasound transducer mounted on the transducer mounting propagate through the ultrasound propagation fluid and through the flexible membrane to the object, and back through the flexible membrane and ultrasound propagation fluid to the ultrasound transducer.

The flexible member and the ultrasound propagating fluid within the reservoir cooperate to accommodate the shape of the object to be scanned in that the external contact surface adapts or conforms to a negative of the shape of the object on which it is placed. In this way, improved contact with a variety of objects can be achieved, which improves the quality of the ultrasound scan. It may also allow for composite ultrasound images of a same region to be obtained as the improved contact mitigates against movement of the transducer relative to the region being imaged between image acquisitions and/or allows for precise placement of the apparatus relative to the object being scanned facilitating scanning of a desired specific region. Furthermore, the ease of effective positioning of the external contact surface of the apparatus on an object to be scanned, as facilitated by the cooperation between the flexible member and the ultrasound propagating fluid, means that the apparatus may be used effectively by untrained operators.

The apparatus of the described embodiments may facilitate the acquisition of high quality ultrasound images by mitigating the likelihood of air gaps between the mounted ultrasound transducer and the object to be scanned, providing the benefits of a fluid bath to the imaging process without need to submerge the object.

The above example illustrating use in meat grading activities is merely one example of an application of the apparatus of the present application. Further examples of activities for which the apparatus 102 may be used to provide an improvement in acquiring ultrasound scans include animal carcass merit assessment, ultrasound image collection for small animals (such in as veterinary use), image collection to inspect for different meat cuts, and genetic selection.

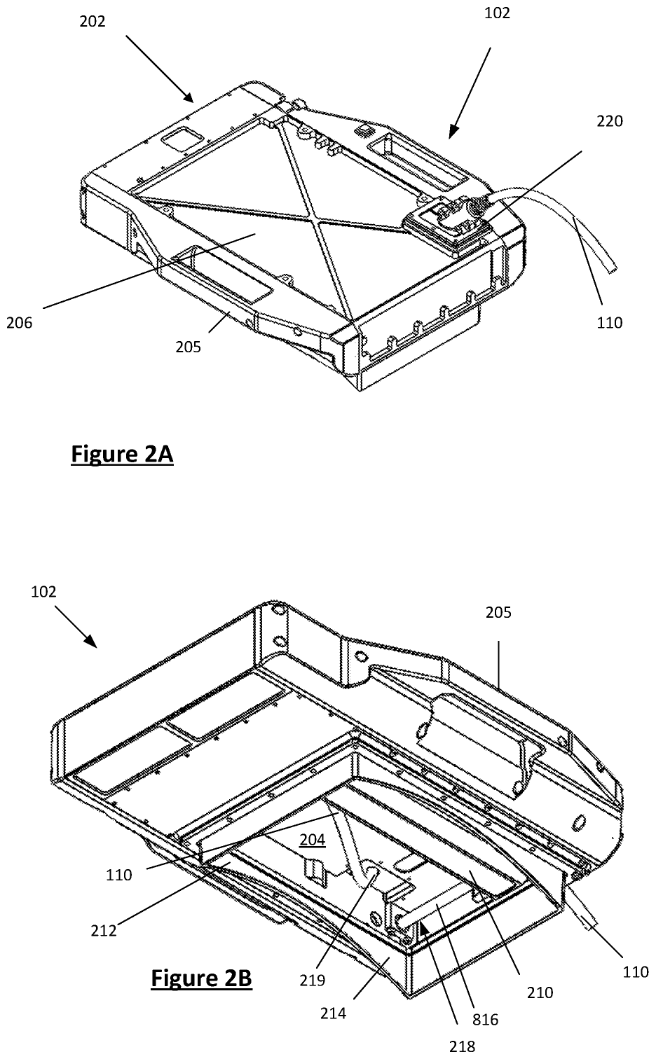

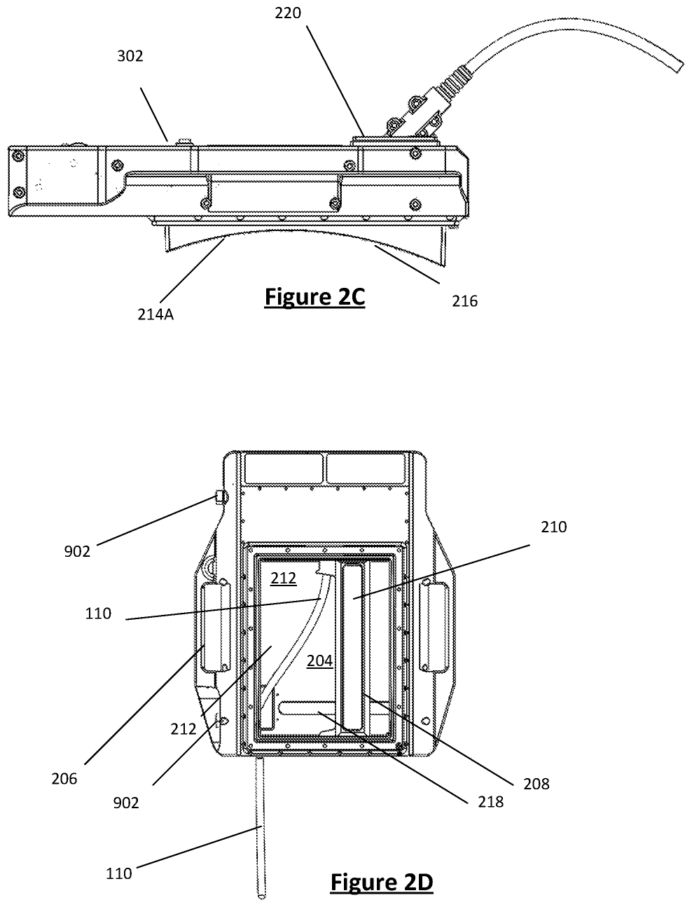

Referring now to FIGS. 2A to 2D, there is shown an apparatus 102 for facilitating ultrasound scanning of objects, according to some embodiments. The apparatus 102 comprises a housing 202 defining a reservoir 204. The reservoir 204 may be arranged to be filled with ultrasound propagation fluid, such as oil or water, as discussed in more detail below.

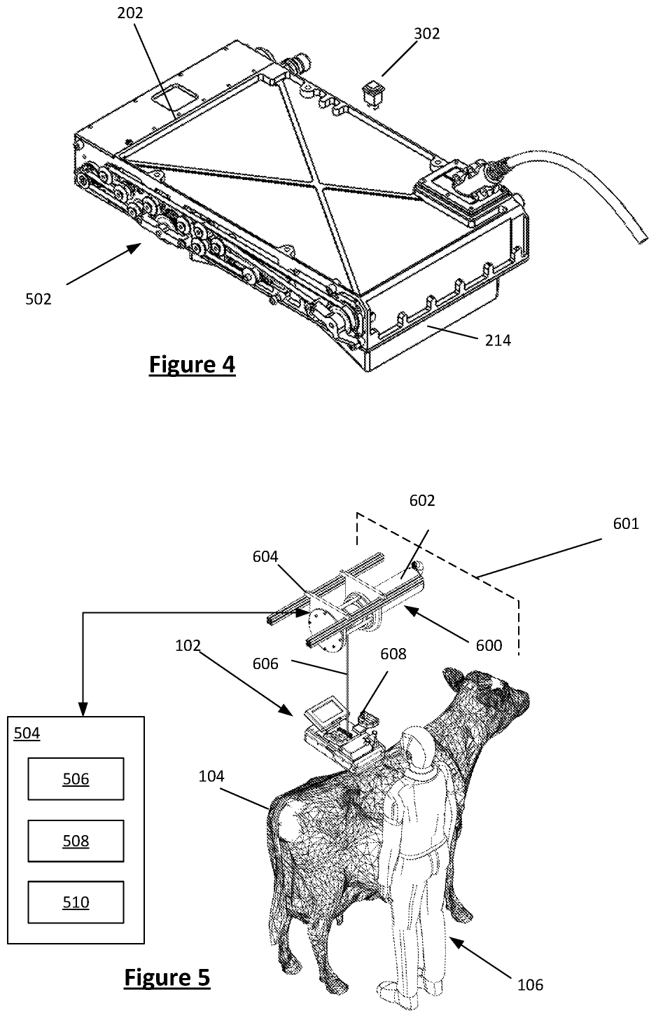

The housing 202 comprises a body 206. As shown in FIGS. 2A, 3B, 4A and 5, for example, the body 206 may be substantially rectangular or square shaped. The body 206 may define a cavity 212 arranged to accommodate a transducer mounting 208 configured to support an ultrasound transducer 210. The body 206 may define an upper or lower wall or end of the reservoir such that the cavity 212 defined by the body 206 is part of the reservoir 204. The transducer may be an ultrasound transducer 210 capable of producing and receiving ultrasound in the range of 0.1 MHz to 20 MHz.

The housing 202 comprises a flange 214 extending from the body 206 to define side walls of the reservoir 204. In some embodiments, the flange 214 comprises or is composed of moulded silicone.

The flange 214 may be connected to, or mounted on the body 206. In some embodiments, the flange 214 may be connected to the body 206 via adhesive. In some embodiments, the flange 214 may be removeably coupled to the body 206. For example, the flange 214 may be coupled to the body 206 using connectors such as screws or bolts, and optionally a gasket comprising apertures to accommodate the connectors. The selective removability of flange 214 from the body 206 may allow for better operational use, allowing cleaning and service of the flange 214, and also allowing for replacement of the flange 214 if broken or damaged, for example.

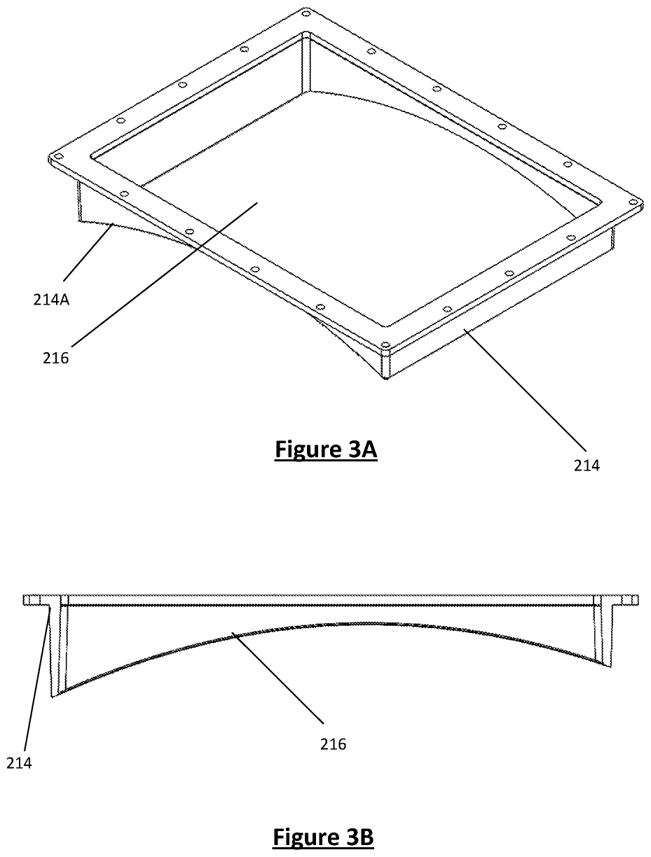

As best illustrated in FIG. 3A, a first end 214A of the side walls of the flange 212 (that is the free or distal end of the flange 214 relative to the proximal end coupled to the body 206) may comprise a substantially curved surface or profile. For example, the first end 214A of the flange 214 may define a substantially concave surface.

The housing 202 comprises a first flexible membrane 216 mounted or coupled to the first end 214A of the flange 214, as illustrated in FIGS. 3A and 3B. In some embodiments, the first flexible membrane 216 is glued or otherwise secured to the flange 214. For example, in some embodiments, a perimeter of an inner surface of the first flexible membrane 216 is secured to the first end 214A of flange 214. In some embodiments, the first flexible membrane 216 and flange 214 are a single piece of silicone. In such embodiments, the first flexible membrane 216 and flange 214 may be cast as one unit. This may negate any need for gluing or otherwise coupling the first flexible membrane 216 and flange 214 together.

The first flexible membrane 216 is configured to allow ultrasound waves to propagate there through. The first membrane 216 forms an external contact surface (not shown) of the housing 202 which is arranged to make contact with an object to be scanned.

The flexible membrane 216 may comprise or may be composed of an elastomeric material, such as silicone. For example, the flexible membrane may be sheet of silicone. The flexible membrane 216 may have a thickness within the range of 0.1 mm to 3 mm. In other embodiments, the flexible membrane 216 may have a thickness within the range of 0.2 mm to 1 mm. For example, the flexible membrane 216 may have a thickness of about 0.3 mm. A flexible member 216 having characteristics of flexibility and elasticity may allow for the membrane to better conform to a curved shape as may be defined by the flange 214. For example, the flange 214 may define a substantially concave shape and accordingly, the flexible membrane 216 secured thereto may conform to the substantially concave shape and thereby define an external contact surface having a substantially concave surface. Such an external contact surface may be appropriate for conforming to or cooperating with a convex shaped object, such as the back of an animal. The flexible membrane 216 may be configured or conformed into a shape that is approximately the negative of an object to be scanned by configuring the shape of the flange 214 accordingly. For example, in the case of scanning the back of an animal which has a convex shape, the flange 214 may be manufactured to have a substantially concave shape so that it accommodates or approximately matches the shape of the objects to be scanned. Additionally, the flexibility of the flexible membrane 216 may allow it to accommodate variations or deviations from the manufactured shape defined by the flange 216, as may be required to accommodate variations in object size of a specific object type, for example, various sizes of animal backs.

The flexible membrane 216 may be comprised of a material that exhibits one or more of the following characteristics: an acoustic impedance similar to that of an object (or objects) for which it is to be used to scan; a relatively low acoustic attenuation rating; flexibility; relative softness; relatively easy to fabricate; relatively robust; relatively easy to clean; relatively safe. In some embodiments, the flexible membrane 216 comprises or is composed of silicone, which provides a number of benefits for the apparatus. The acoustic impedance of silicone is similar to that of hide, animal skin, fat, oil, and muscle. The acoustic attenuation of silicone is also relatively low. As blood, soft tissue, muscle, fat, and water have impedances of a range between 148-170 KRayl, a material choice for the flexible member that has a similar acoustic impedance is beneficial. The acoustic attenuation of the flexible membrane 216 may be approximately 1-6 dB/mm. Silicone has a relatively low acoustic attenuation which when coupled with its mechanical properties enables it to be used as a relatively thin membrane with very low acoustic attenuation. Because attenuation is thickness dependent the thinner the membrane the less attenuation occurs. Accordingly, when the apparatus 102 is used for performing ultrasound scanning of animals, the use of a flexible membrane 216 comprising silicone provides for effective ultrasound signal propagation. Silicone is flexible, allowing the flexible membrane 216 to accommodate the shape of an object to be scanned. Silicone is soft, in some embodiments having a shore value of about 20-80A. Softness ensures contact with objects 104, such as animals, is not uncomfortable or hazardous, which has a dual advantage of ensuring animal welfare as well as minimizing agitation and subsequent movement of the animal that can degrade the quality of the captured ultrasound data. Furthermore, softness helps with providing an even contact with an object or across a target specimen, allowing for relatively consistent ultrasound propagation. Silicone is an easy material to form into a complex shape using simple molding techniques. The process of making the curved contour is also highly accurate and repeatable as the same mold can be used repeatedly. Silicone is a very resilient material able to withstand 350-500% elongation before breaking whilst also maintaining structural integrity over a wide temperature range. Furthermore, the material strength of silicone means that a thin layer is usable many times while not impacting ultrasound signal attenuation. Silicone can be extremely difficult to adhere to, making its cleaning after use in oily and dusty environments straightforward. Silicone can be easily purchased in a variety of grades, from skin safe to food safe. Furthermore, it is a widely available material. It will be appreciated that alternative materials, other than silicone, may be used.

Referring to FIG. 2D, the transducer mounting 208 may be provided or disposed on the body 206 such that the ultrasound transducer 210 supported by the transducer mounting 208 is received within the cavity 212 of the body 206. In some embodiments, the ultrasound transducer 210, when mounted on the transducer mounting 208, extends along a length of the cavity of the body 206. For example, as shown in FIG. 2D, the ultrasound transducer 210 may span the cavity 212. The flexible membrane 216 is displaced from or spaced-apart from the body 206 by the flange 214. When the flexible membrane 216 is coupled or mounted to the flange 214, as shown in FIG. 2C, the ultrasound transducer 210 is orientated such that it faces, or is opposite to the flexible membrane 216. Accordingly, when activated, and the reservoir 204 is filled with ultrasound propagation fluid, ultrasound waves emitted from the ultrasound transducer are propagated towards, and through the flexible membrane 216.

In some embodiment, the transducer mounting 208 and accordingly the transducer may be mounted or disposed on an external surface of the housing 202, such that the transducer 210 is not provided within the reservoir or in contact with the ultrasound propagation fluid provided therewithin. In such embodiments, the housing 202 may comprise a second membrane (not shown) capable of allowing ultrasound waves to pass therethrough. The transducer mounting 208 may be configured to position or orientate the transducer 210 at the second membrane (not shown) (and external to the reservoir 204) such that an ultrasound wave being emitted from the transducer 210 passes through the second membrane (not shown), into the reservoir 204, and through the flexible membrane 216 to the object being scanned. For example, the second membrane (not shown) may be located opposite the flexible membrane 216. The transducer 210 may be disposed on the second membrane. In some embodiments, a propagating fluid may be located on an external surface of the second membrane to facilitate efficient ultrasonic communication between the ultrasound transducer 210 and the second membrane.

The transducer mounting 212 may be configured to cooperate with a track or bulkhead 218, such as an elongate member or bar. The track 218 may span a first width of the cavity 212 of the body 206. The track 218 may be configured to extend through an aperture 219 disposed through a first end of the transducer mounting 208 such that the transducer mounting 208 and accordingly a transducer 210 mounted thereto, can be moved from a first side of the cavity 212 to a second opposite or facing side of the cavity 212 along the track 218. In some embodiments, the body 206 may comprise a second track (not shown), such as an elongate member or bar. The second track (not shown) may span a second width of the cavity 212 of the body 206. For example, the second width may be orthogonal to the first with. The transducer mounting 208 may comprise a complimentary aperture (not shown) configured to receive the second track there through, and allow the transducer mounting 208, and accordingly a transducer 210 mounted thereto, to be moved from a third side of the cavity 212 to a fourth opposite or facing side of the cavity 212 along the second track 218.

The transducer mounting 208 being moveable along the track 218 allows for ultrasound measurements to be taken across a range of positions along the track 218 relative to the contact surface 204. In some embodiments, the transducer mounting 208 is moveable in at least two directions within the housing 202. In some embodiments, the transducer mounting 208 includes a motor system 502 to move the transducer 210, such as a stepper motor or servo motor. In some embodiments, screws (such as lead screws) can be turned by the motor to move the transducer mounting 208 in a direction along a shaft, such as the track 218.

In embodiments where the transducer mounting 208 is moveable, care must be taken to preserve the environmental seal of the housing 202 to avoid entrance of air into the reservoir 204. In some embodiments, a magnetic coupling is used to allow torque to be transmitted from a coupler disposed outside of the reservoir 204 to a coupler disposed within the reservoir 204, without a physical connection between them. If instead a physical connection was provided between the reservoir 204 and the exterior of the housing 202, it may rely on O-ring seals being provided on shafts. Such O-rings tend to wear over time and need replacement, which is not ideal for systems that have very high duty cycles. O-rings create a significant amount of friction and would therefore need a much larger and more powerful motor to overcome this force adding significant weight and size to the apparatus 102. Though relatively robust, the dynamic nature of a shaft O-ring seal also means that very small amounts of air are still capable of getting past the seals mount, which is attached to screws or lead screws, and entering the reservoir 206.

The driving mechanism 800 comprises a pair of magnetic couplers 804, 806. The magnetic couplers 804, 806 may comprise axially magnetized neodymium magnets (not shown) disposed in an array around a perimeter of each coupler. In some embodiments, the magnets (not shown) are arranged such that the poles of neighbouring magnets in the array alternate. When the couplers 804, 806 are placed opposite each other, for example on either side of a wall of housing 202 (for example one being disposed within the reservoir 204 and the other disposed on an external wall of the reservoir 204), the alternating magnetic field that exists between corresponding magnets rigidly locks the couplers 804, 806, allowing torque to be transmitted without a physical connection between them. For example, the magnetic couplers 804, 806 may be arranged such that a positive pole of the magnetic couplers 804 is configured to be opposite or face a negative pole of the magnetic couplers 806.

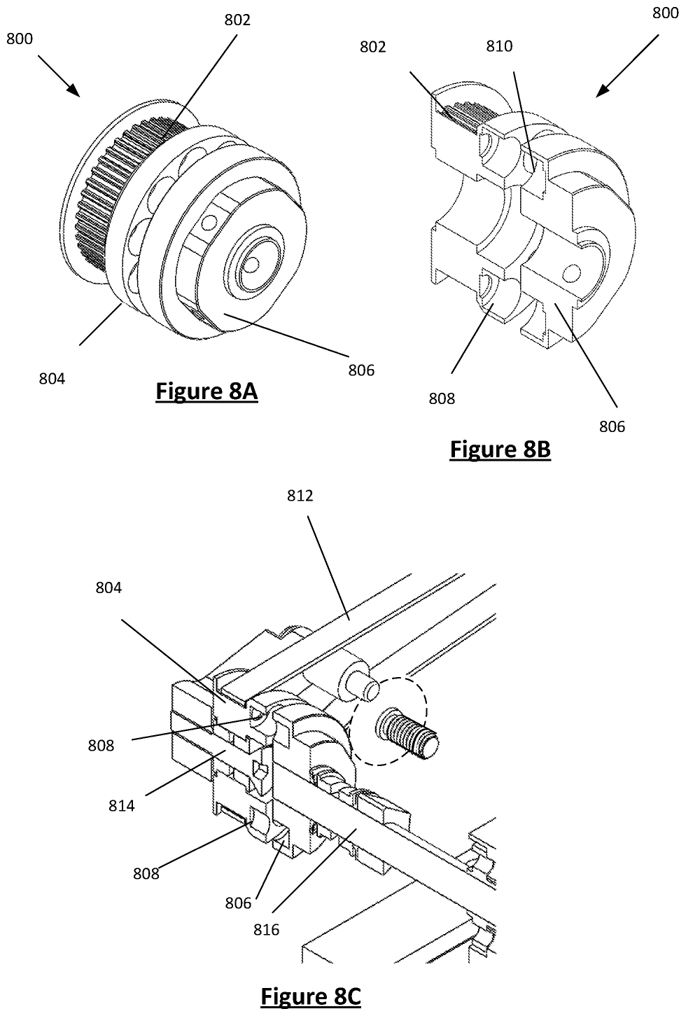

FIGS. 8A to 8C depict the driving mechanism 800 in greater detail. The driving mechanism 800 comprises a motor coupler 804 and shaft coupler 806. In some embodiment, the motor coupler 804 comprises a coupling ring having belt teeth 802 arranged or disposed around its circumference to engage a motor belt 812. Magnet receiving slots 808 are arranged or disposed about the perimeter of each coupler 804, 806, each slot 808 being configured to receive at least one magnet—as shown in FIG. 8B. The receiving slots 808 of motor coupler 804 are configured to match or cooperate with respective number of the receiving slots 810 of the shaft coupler 806 such that when magnets are loaded into the slots in opposing polarities on the motor and shaft sides, the magnetic attraction is sufficient to provide torque when a motor axle 814 is turned, providing a rotational effect to the shaft 812 without need for direct physical contact. The transducer mounting 208 may be connected to shaft 816 to allow for translation of the transducer mounting 208 along the length of the shaft 816 when the shaft is rotated. In such embodiments, the shaft 816 comprise a lead screw (or leadscrew) with a matched fitting on the transducer mounting 208. The arrangement of magnets in the motor coupler 804 may comprise an alternating facing of positive and negative around the circumference. This alternating arrangement may provide a stronger rotational force to the shaft coupler 806 compared to a polarity-per-coupler approach, whereby each of the couplers has magnets facing in one opposing polarity.

The use of magnetic coupling within the apparatus 102 provides beneficial effects through reducing need for maintenance by providing an improved functionality due to the magnetic coupling being a non-wearing component. The specific configuration of the motor coupler 804 having belt teeth 802 around its circumference allows for a reduced profile of the motor system 502, and according of the apparatus 102 as a whole.

In some embodiments the motor may be a stepper motor. The use of a stepper motor or servomotors allows for position identification of the transducer mounting 208 without relying on additional sensors, which would likely add bulk and provide possible points of ingress for air to the reservoir 204 of the housing 202. The use of the motors at the exterior of the reservoir 204 of the housing 202 means that the only cable entering the reservoir 204 is the cable 110 supplying power and data transfer to the mounted ultrasound transducer 208.

In some embodiments, the motor may provide a torque of up to 40 Ncm which effectively rotates the shaft coupler 804 in the described arrangements. In some embodiments, the motor may be a Nema 14 stepper motor. In some embodiments, two motors are provided to provide movement of the transducer mounting 208, and accordingly the transducer 210, in two different direction (a first direction and a second direction), one of which may traverse along a length of an elongate external contact surface, the other which may traverse along a width of the elongate external contact surface in a direction substantially orthogonal to the first direction. In such embodiments, the two motors may be arranged or disposed on an external surface of the housing, and which be on a same side or surface of the housing 202 to minimize the space taken up by the motor system 502. In some embodiments, the motors may be arranged on opposing sides of the housing 202.

In order to avoid slippage due to high torque, a maximum required motor torque for motion may be less than the slipping torque of the coupler. The slipping torque is a function of how far the couplers 804 and 806 are from each other. For example, in testing, a coupler was capable for transmitting the required torque with a gap of up to approximately 3.5 mm. In some embodiments, there is approximately a 1.2-1.8 mm gap between the two halves and the mechanism 800 has operated reliably without failure. The arrangement 502 depicted in FIGS. 5A and 5B further depicts the arrangement of the motor and track, enabling the transducer mounting 208 to be moveable. This arrangement allows for a broader scanning region to be imaged by the apparatus 102 during use.

The transducer mounting 208 may further comprise a cable support 220 to allow cable/s 110 to supply power to apparatus 102 and/or an ultrasound transducer 210 received in the transducer mounting 208. The transducer mounting 208 may further comprise a cable mounting gasket 222 configured to retain the cable 110 within the transducer mounting 208, and a cable gland 224. The cable gasket 222 and cable gland 224 cooperate to allow an effective environmental seal for the interior of the housing 202 from the external environment, and from air ingress

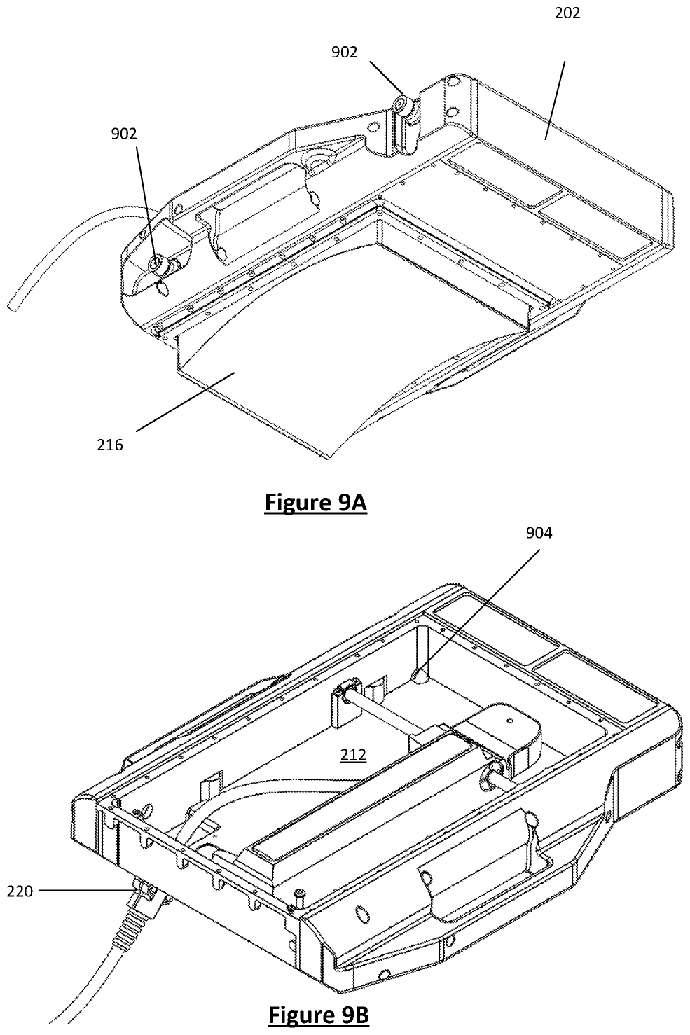

The housing 202 may further comprise one or more fluid ports 902. FIGS. 9A and 9B depicts first and second fluid ports 902 being disposed on a same side of the housing 202, allowing a user 106 to add, fill or drain propagation fluid from within the housing 202. In some embodiments, the fluid port(s) 902 may comprise dry break fittings, or other fittings requiring a physical activation to open. The use of such fittings mitigates the chance of air entering the housing 202 during a filling operation. The fluid port(s) 902 may comprise combined fluid inlets and outlets. In some embodiments, the each of the fluid ports 902 may be configured to act as either an inlet or outlet. For example, the first port may be an ingress port and the second port may be an egress port. The interior of housing 202 may further comprise air traps 904. The air traps 904 may comprise recessed slots within a region of the cavity 212 of the body, for example, at the level of and adjacent to the fluid ports 902. In some embodiments, the air traps 904 allow for trapped air to rise to the level of a fluid port 902 when the apparatus 102 is held in an upright position, allowing for easier removal of trapped air through the fluid port 902. To fill the reservoir with propagation fluid, the apparatus may be arranged such that the first port 902, that is the ingress port or inlet, is orientated to be at a highest position.

The effective sealing of an ultrasound transducer 208 within the housing 202 allows for the minimisation or elimination of air within the reservoir 204. Any air present between the ultrasound transducer 208 and the object 104 significantly degrades the quality of the ultrasound signal. In the present embodiments, the sealing of the gasket 118 and cable gland 216 mean that air which would enter the housing 202 interior, through cable mountings, is effectively mitigated.

In existing solutions, to seal a cable the accepted standard is to use a cable gland. However using a cable gland is dependent on at least one end of the cable being the same size or smaller than the opening of the cable gland otherwise it is not possible to insert the cable through the gland. In some embodiments, the apparatus 102 may have gasket 118 in-situ on the cable 110 itself. In creating such a gasket, a two-part mold may be used, with the negative shape of the required sealing gasket, which is then bolted around the cable 110, which is then filled with silicone. The silicone then creates the shape of the gasket 118 required to seal against the body of the apparatus 102, whilst creating an airtight cylinder around the cable 110, which can then be secured once it has cured.

In some embodiments, a gland seal (not shown) may be included on cable 110 during the manufacturing process. In some embodiments, flat sealing gaskets may be used to provide a more effective environmental seal for the apparatus 102, across various potential entry points on the apparatus.

The housing 202, including the flexible membrane 216, cooperate to define the reservoir 206 that is configured to retain a volume of ultrasound propagation fluid between the transducer mounting 208 and accordingly the transducer, and the flexible membrane 216. In use, this reservoir may be filled with a propagation medium. The reservoir may be configured to retain approximately 3-4 litres of propagation fluid. In some embodiments, the fluid capacity of the reservoir may be larger, and configured to receive and/or store, such as 4 litres, or up to 5 litres. In some embodiments, a small volume of fluid may be retained, such as between 1-3 litres. The sealing of the apparatus described above provides a substantially air free, environmentally sealed environment. Where the transducer mounting 208 and the transducer 208 are disposed within the cavity 212 of the body 206 of the housing 202, the transducer 208 may be substantially submerged in the propagation medium. It is this contact surface 204 which keeps the fluid inside of the apparatus 102 in use, and during scanning the contact surface 204 is pressed against the external target 104. In use, a thin layer of propagation fluid is applied to the external target 104 to better provide consistent contact. This fluid can be ultrasound fluid/gel, oil or water. A common example of such ultrasound fluid/gel is the clear gel used during pregnancy checks. In some embodiments, the propagation fluid applied to the surface of the external target 104 and inside the reservoir defined by the housing 202 is the same.

In some embodiments, the propagation medium or ultrasound propagation fluid may be or comprise oil. Oil, for example, a vegetable oil, such as canola oil or sunflower oil, may be used in situations where the object being scanned is a live animal. This is because the ultrasound signal needs to penetrate the animal's hair. As such oil may be applied to the back of the animal to establish good contact between the external contact surface of the apparatus 102 and the hide, even with hair in the path of the ultrasound. Water in these situations is not as effective because the water does not penetrate animal hair.

In some embodiments, the propagation medium or ultrasound propagation fluid may be or comprise water. In an environment such as inside an abattoir where the hide has already been removed, it may be preferable to use water. For food safety reasons, water may be a preferable choice in case the flexible membrane 216 fails. Where water is used as the ultrasound propagation fluid within the reservoir 206, water can be used directly on the back of the animal/carcase to facilitate good contact with the external contact surface of the flexible membrane 216.

The housing 202 may further comprise operational switch 302, comprising a push button or other switch type device, allowing simple on/off operation by a user 106.

The apparatus 102 may further comprise handle(s) 205, to enable a greater ease of use by a user 106 when taking ultrasound measurements. For example, a handle 205 may disposed on an external surface, such as a longitudinal side of the housing 202 of the apparatus 102. In some embodiments, a handle 205 may extend outwardly from each longitudinal side of the apparatus 102, as shown in FIGS. 2A, 2B and 2D, for example.

FIG. 5 illustrates a scanning system 600 comprising the apparatus 102 connected to a safety mechanism or winch 600 via a winch mount 608, according to some embodiments. The winch 600 comprises a shaft 602, motorised pulley 604, and cabling 606.

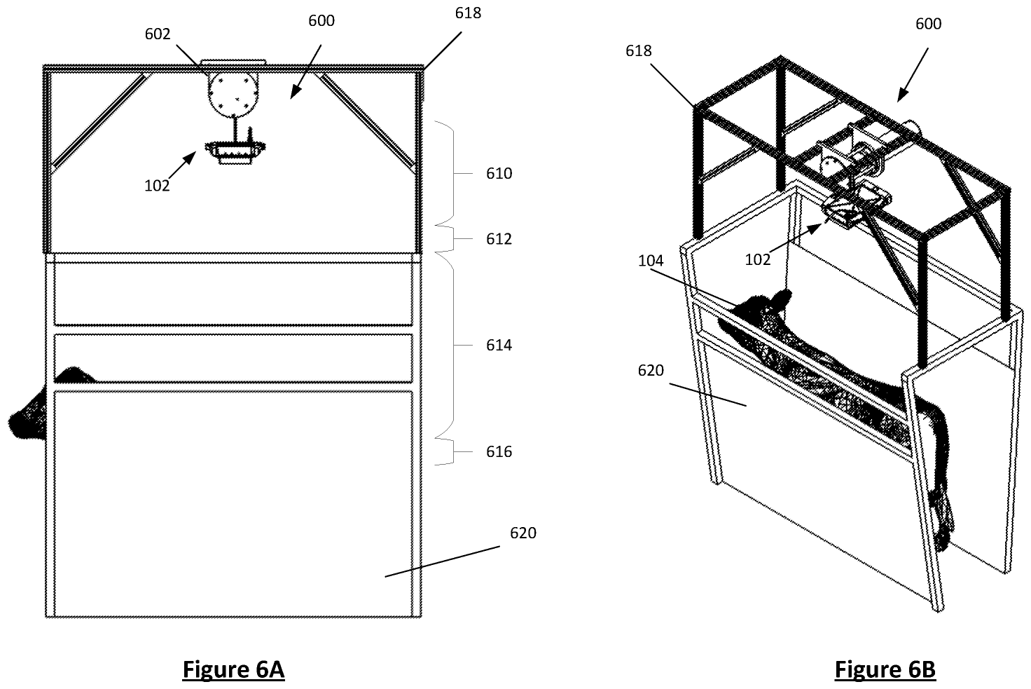

FIGS. 6A and 6B depict a cattle enclosure 620 comprising a frame 618. The scanning system 600 is shown mounted to the frame 618. The scanning system 600 comprises the apparatus 102 and the winch 602. The shaft 602 may be affixed or coupled to a structure or frame 618 to support the pulley 604 and cabling 606, as shown in FIGS. 6A and 6B. The pulley 604 may be a motorised pulley, which may be activated to rotate in a first and/or second direction, to cause attached cabling 606 to be raised or lowered. The cabling 606 may be affixed to the pulley 604 and shaft 602, and be of sufficient tensile strength to support at least the weight of the apparatus 102. In some embodiments, the winch 602 may be configured to support up to 100 kg. In some embodiments, the winch 602 may be configured to support less than 100 kg. In other embodiments, the winch 602 may be configured to support between 100 kg to 200 kg. The frame 618 may be disposed above the cattle enclosure 620 to assist in mounting the winch 602 over an object to be scanned, such as an animal, located within the enclosure 620.

The winch 600 may allow a user 106 to have a greater ease of use of the apparatus 102 during a scanning operation by supporting the weight of the apparatus 102 at a fixable height. Accordingly, a user 106 using the apparatus 102 in this arrangement can selectively release the apparatus between different external targets 104, or set the apparatus 102 at a specified height during scanning. The winch 600 therefore may assist to lower the fatigue of a user 106, reduce operational health risks, and/or create a more consistent scanning procedure.

The scanning system 600 may comprise a controller 504 (FIG. 5) configured to operate the winch 600. The controller 504 may comprise one or more processors 506 and memory 508 storing computer executable instructions, which when executed by the processor(s) 506, cause the winch 600 to operate in accordance with one or more pre-defined settings or thresholds. In some embodiments, the controller 508 comprises a user interface 510 to receive values for the settings(s) from a user. For example, the settings or pre-set thresholds may define or relate to different zones, on/off commands, emergency stop commands, and/or other operation information.

In some embodiments, the scanning system 600 may track the height of an attached apparatus 102 by the rotation of the motorised pulley 604. Motorised pulley 606 may communicate its rotational position with the controller 504 to allow for a user 106 to define different extension positions of the apparatus 102 which correspond to operating zones. The operating zones may comprise regions where the apparatus 102 is held in position, allowed to be moved freely with its weight supported by the winch, or to be moveable with resistance. The resistance to a user 106 raising and lowering the apparatus 102 when connected to the winch 600 may be provided by the motorised pulley 606, to apply more or less resistance based on which operating zone the apparatus 102 is within. The operating zones may be based on a region between a maximum height of the shaft 602 at the installation site, and a minimum height of the expected height of a scanning target. A user 106 may program the winch 600 to define the maximum and minimum heights, and the operating zones between them. In some embodiments, the operating zones are based on a pre-set percentage distribution between a set maximum and minimum height. In some embodiments, the operating zones are set by a user 106.

The user 106 may use an actuator, such as a switch or toggle switch, located on apparatus 102 or as part of a fixture of the scanning system 600, to selectively move the apparatus 102 up and/or down from a definable safe position or height. This may be at the maximum retraction of apparatus 102 on the winch 600, ensuring that the apparatus 102 is at a safe distance when a new target animal 104 enters the area to be scanned. An operator 106 may then toggle the switch to lower the device to a latching position, proximal to the external target 104. At this point, the operator 106 can physically pull the apparatus 102 into a designated ‘weightless’ height, where the apparatus is not locked at a fixed height by the winch 600, but the weight of the apparatus 102 is being supported by the winch 600. At any point, an automatic stop may be connected to the winch 600 to cut all power, acting as a safety brake. If power is lost, the safety brake feature of the automatic stop may prevent the device from falling.

FIG. 6A depicts a number of operating zones 610, 612, 614, and 616. In some embodiments, a first rigid safe zone 610 may be defined from the maximum winch height of a region of approximately 200 mm. As described above, this (and subsequently described operating regions) may be a user defined region based on the known installation height of the winch 600. The rigid safe zone requiring a large amount of force to overcome, for example 30 or more kilograms of weight being applied to the winch 600. This zone prevents users from moving the device lower than allowable.

A latch zone 312 may be then defined at the bottom of the first rigid zone, of a region of approximately 1-2 mm, wherein the apparatus 102 is held at a fixed height until a user pulls the apparatus 102 into a weightless zone.

A weightless zone 614 may be defined at the bottom of the latch zone, of a region of approximately 650 mm, wherein the weight of the apparatus 102 is supported entirely by the winch 600—providing a ‘weightless’ feeling to the user 106.

A second rigid zone 616 may be provided at the bottom of the weightless zone, of an indefinite length up to the maximum extension of the winch 600. The second rigid safe zone requiring a large amount of force to overcome, for example 30 or more kilograms of weight being applied to the winch 600. This zone prevents users from lowering the device lower than allowable.

In other embodiments, other zone ranges may be used to adapt the winch 600 and apparatus 102 to different environments and installations, thereby allowing a greater range of flexibility in installation and ease of use for users 106. The size of the zones may be customizable for different installations, allowing an adaptable winch system 600 useable in a wide range of installation sites.

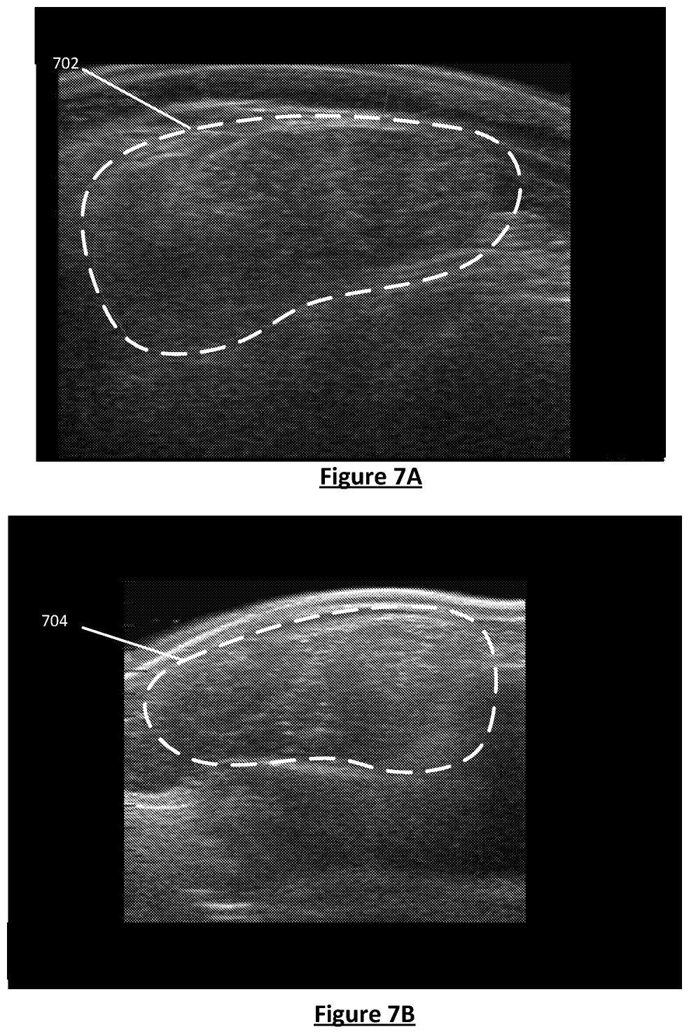

FIG. 7A depicts an ultrasound image of a region 702 of a live animal using an existing apparatus which comprises an ultrasound transducer mounted in a transducer standoff. FIG. 7B depicts an ultrasound image of the same region, 704 as captured using the apparatus 102 of the described embodiments. As depicted, the image captured using the apparatus 102 of the described embodiments has improved resolution, clarity, and definition. The described embodiments therefore may provide an ultrasound measurement device providing a more repeatable, reliable and efficient enabling accurate data capture at a scale not previously possible by known solutions.

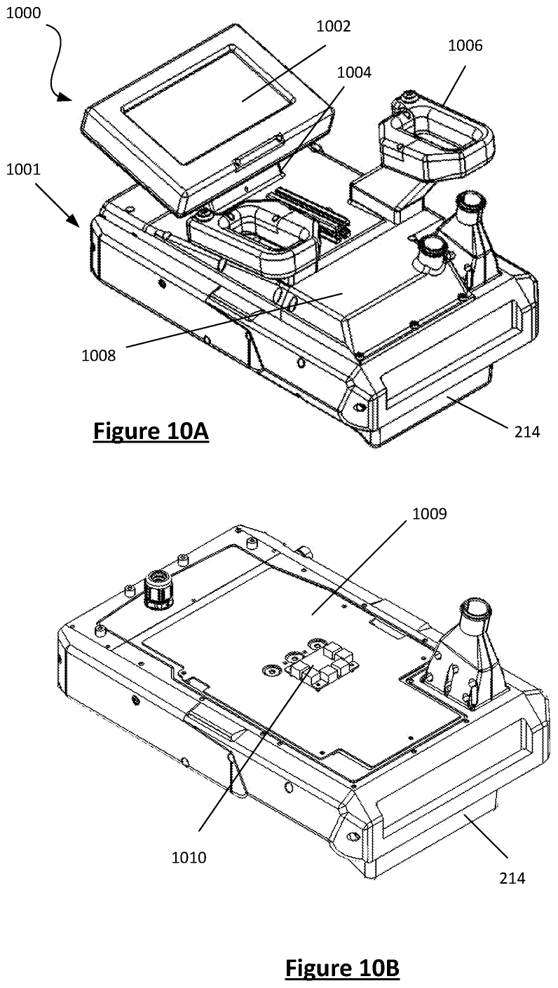

Referring now to FIG. 10A, there is depicted a top perspective view of an apparatus 1000 for facilitating ultrasound scanning, according to some embodiments. The apparatus 1000 may comprise any or all of the features and components of apparatus 102 described above.

The apparatus 1000 comprises a housing 1001. The housing 1001 may comprise a display screen 1002. The display screen 1002 may be attached to a backing plate 1008 via a screen mount 1004. Display screen 1002 may be configured to receive output from the transducer 210 during scanning, and display the ultrasound image output to a user 106. The display screen 1002 may further comprise a controller, and operate as previously described client device 108. The display screen 1002 may be configured to receive ultrasound data from ultrasound device 210, and display ultrasound images of targets 104, such as live ultrasounds image of targets 104 when the apparatus 1000 is in use. This live feedback may provide for more accurate usage of the apparatus 1000, enabling a user 106 to adjust the position of the apparatus 1000 relative to a target 104, for example, to more easily identify desired regions, and/or to correct for positional errors in scanning. In such embodiments, display screen 1002 may be the display of a smartphone device, tablet PC, or other computing device. In other embodiments, the processing of the ultrasound data occurs on an external client device 108, as previously described, and the processed image is displayed on display screen 1002.

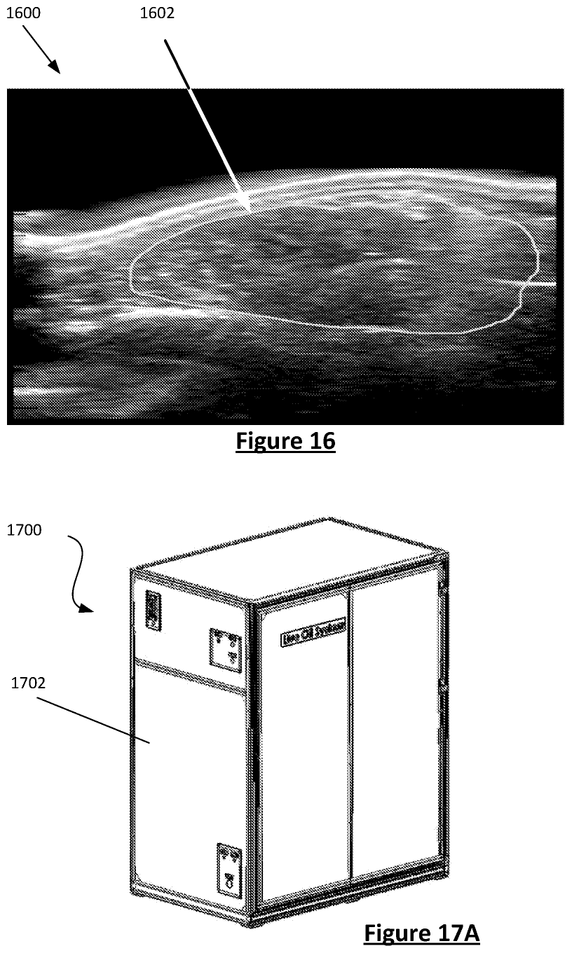

FIG. 16 is an example ultrasound image captured by the apparatus 1000 and displayed on display screen 1002. Region 1602 may be highlighted by an overlayed graphic, being drawn live by processing software on client device 108, and displaying an identified muscle area. In such embodiments, client device 108 may comprise muscle area identification software to determine the location of a desired muscle area in an image frame, and then highlight the identified muscle area in the image frame. In some embodiments, display screen 1002 may comprise a mobile or tablet device. In some embodiments display screen 1002 may be an LCD display.

The apparatus 1000 may comprise one or more handles 1006. Handle(s) 1006 may connect directly to the backing plate 1008. Handle(s) 1006 may extend outwardly from the backing plate 1008 of the housing 1001. The position of handle(s) 1006 may allow for an improved ease of use of the apparatus 1000, and may enable a greater degree of control of the apparatus 1000 when placing it against a target 104 by a user 106.

Backing plate 1008 may comprise a removable cover, allowing access to the internal components of apparatus 1000. Backing plate 1008 may be a 3D printed removable cover. Backing plate 1008 may contain apertures or mounts, suitable to affix display screen 1002 to the housing 1001. Backing plate 1008 may comprise mounts for handle(s) 1006.

When ultrasound scanning is being performed, a mismatch between the temperature of the propagation fluid provided within apparatus 102, 1000, and fluid provided on a surface of the target to be scanned may result in an impedance mismatch that is significant enough to negatively impact the ultrasound image being captured. This may be particularly problematic where the apparatus 102, 1000 is being used during the colder months, or in cold climates. For example, the impedance mismatch between cold propagation fluid in the reservoir 204 and warmer propagation fluid on a prepared surface of a target 104 can cause ultrasound waves to be reflected back before penetrating the target 104. This may result in unclear ultrasound images of the target 104 being acquired. If the impedance mismatch is too great, a completely black image may be obtained. In such conditions, the ultrasound waves do not penetrate the target 104. To overcome or mitigate such problems, the apparatus 1000 comprises a heating control system 1012 configured to selectively heat the propagation fluid disposed within the reservoir 204 of the apparatus 1000. Heating control system 1012 may comprise controller 1104. By selectively heating and/or maintaining the propagation fluid within a particular temperature range, for example, relatively close to body temperature, such as a 25-45° C. temperature range, the temperature of the propagation fluid will more likely better match the temperature of the target or animal being scanned, and the temperature of any fluid provided on the surface to the target. In some embodiments, the temperature range is narrower, such as between 32-44° C. In some embodiments, the temperature range may be 43-45° C. In other embodiments, suitable temperature ranges are used, in order to reduce or mitigate any impedance mismatch. Accordingly, any impedance mismatch may be reduced and the ultrasound waves emitted from the apparatus 1000 may pass through an interface layer of propagation fluid on the target 104, to allow an ultrasound scan of the target 104 be obtained with minimal or no reflection.

The apparatus 1000 comprising the heating control system 1012 is described now with reference to FIG. 10B to FIGS. 15. In some embodiments, the heating control system 1012 comprises a power source 1013, one or more heating elements 1102, one or more temperature sensors 1108 and a controller 1104.

FIG. 10B is a top perspective view of the apparatus 1000, with backing plate 1008 of FIG. 10A removed, according to some embodiments. As illustrated, and in some embodiments, a power distribution circuit 1010, such as a power distribution printed circuit board (PCB) is provided within the housing 1001 of the apparatus 1000, for example, on a backing circuit board 1009, such as a PCB, which allows power to be distributed from the power distribution circuit to components of the apparatus 1000, such as the heating elements 1102, and in some embodiments, a pump motor system 1106, as discussed in more detail below. Backing circuit board 1009 may act as an interface between the internal and external components, or as a cover for the internal components of the apparatus 1000. Backing circuit board 1009 may act as a thermal barrier, to reduce heat loss from the apparatus 1000. In some embodiments, backing circuit board 1009 may include, or be mounted on, insulation foam, to reduce heat transfer from heating elements 1102 to the external components of the apparatus 1000. Power distribution circuit 1010 may be a 12V distribution circuit and may comprise one or more power sources 1013, such as one or more 120 W 12V dc power source. In the embodiment depicted in FIG. 10B, the apparatus 1000 comprises two power sources to supply power to the components of the apparatus 1000. The power distribution circuit 1010 may sit atop or be deployed on one or more standoffs to reduce or minimise transfer of heat from the backing circuit board 1009 to the power distribution circuit 1010.

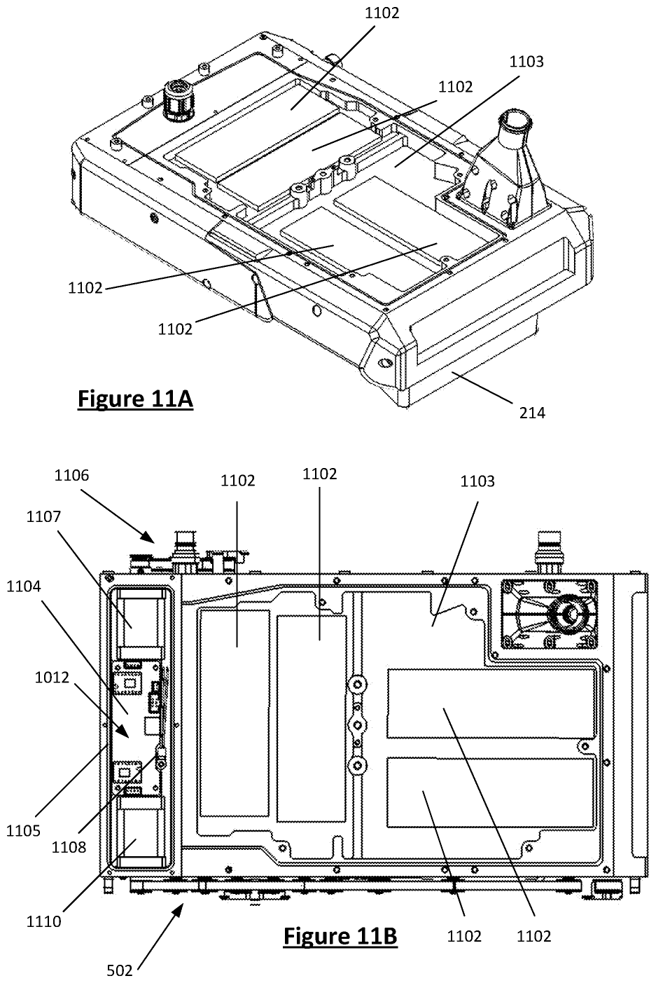

FIG. 11A is a top perspective view of the apparatus 1000, showing a partial internal view. In particular, FIG. 11A depicts a view of the apparatus 1000 with backing circuit board 1009 and power distribution circuit 1010 removed. As illustrated, the apparatus 1000 comprises a plurality of heating elements 1102 arranged within the apparatus 1000.

Heating elements 1102 may comprise silicone heat pads. Heating elements 1102 may be adhesively attached to the apparatus 1000. Heating elements 1102 may contact or may be disposed on a heat transfer surface 1103 of the apparatus 1000. For example, the heat transfer surface 1103 may comprise or correspond with a wall of the body 206 defining the reservoir 204. This may allow for improved heat transfer from the heating elements 1102 to ultrasound propagation fluid stored within the reservoir 204 of apparatus 1000. The heat transfer surface 1103 may comprise a metallic portion, such as an aluminium portion, of the housing 1001. In some embodiments, other suitable heating means may be provided, such as wire heating elements or film heating elements.

In some embodiments, immersion-heating elements may be deployed within the reservoir 204 of the apparatus 1000 to thereby heat the propagation fluid directly. In some embodiments, one heating element 1102 is used. In some embodiments, two heating elements 1102 are used. In some embodiments, three heating elements 1102 are used. In some embodiments, four heating elements 1102 are used. In some embodiments, more than four heating elements 1102 are used.

The temperature sensors 1108 may be placed at one or more locations throughout the apparatus 1000 to allow for a range of temperature readings to be captured. In some embodiments, the temperature sensor(s) 1108 comprise two thermistors. In some embodiments, temperature sensor(s) 1108 are placed proximate to one or more of the heating elements 1102, and temperature sensor(s) 1108 are placed distally from the heating elements 1102, such as toward an upper surface of the body or the housing, for example relatively close to a controller 1104. The temperature sensor(s) 1108 may send or transmit temperature data to a controller 1104. Using the temperature data from the temperature sensor(s) 1108, controller 1104 may determine whether the heating elements 1102 should be turned on or off. The control of the heating elements 1102 and feedback from the temperature sensors 1108 may allow for precise control over propagation fluid temperature. In some embodiments, the temperature of the propagation fluid is controlled to be within a temperature range. The temperature range may be 25-45° C. The temperature range may be within 20° C. of a target temperature. The temperature range may be 5° C. above or below a target temperature. The target temperature may be an expected temperature of a target to be scanned. The expected target temperature may be body heat of a livestock animal, such as a cow, for example. In some embodiments, the temperature range is narrower, such as between 32-44° C. In some embodiments, the temperature range may be 43-45° C. In other embodiments, other temperature ranges may be used.

FIG. 11B is a bottom sectional view of the apparatus 1000. As depicted in this view, controller 504, 1104 may be installed within the housing 1001 of apparatus 1000. For example, controller 1104 may be located within an electronics bay 1105 at or toward an end of the apparatus 1000. As illustrated, at least one temperature sensor 1108, and/or motors 1107, 1110 may be provided within the electronics bay 1105. Controller 1104 may control the operation of the motor systems 502, 1106, motors 1107, 1110 (discussed below), the heating elements 1102, and/or receive temperature data from temperature sensor(s) 1108. In some embodiments, controller 1104 may control the operation of the heating pads 1102.

Similar to controller 504, controller 1104 may comprise one or more processors 506 and memory 508 storing computer executable instructions, which when executed by the processor(s) 506, causes the controller 1104 to control the temperature of the propagation fluid within the apparatus 1000. In some embodiments, controller 1104 is configured to receive temperature data from the temperature sensors 1108 indicative of the temperature of the propagation fluid within the apparatus 1000. In some embodiments, where temperature data is receive from multiple temperature sensor(s) 1108, the controller 1104 may determine the temperature of the propagation fluid within the apparatus 1000 to be an average of the temperature data of the multiple temperature sensors 1108. In some embodiments, one or more temperature sensors may be considered as being more representative of the temperature of the propagation fluid than others, and in such embodiments, the controller 1108 may weight temperature data from those temperature sensors 1108 more heavily than temperature data from the other temperature sensor(s). The controller 1108 may be configured to compare the temperature of the propagation fluid to one or more temperature thresholds. For example, memory 508 may comprise one or more set points. A first set point may correspond with a lower threshold value and a second set point may correspond with an upper threshold value. In some embodiments, responsive to the controller 1104 determining that the temperature of the propagation fluid is less than the first set point, the controller 1104 may be configured to cause power to flow from the power source(s) 1013, for example, by transmitting a signal to the power distribution circuit 1010, to allow power to flow to the heating element(s) causing them to turn on and start to heat the propagation fluid. In some embodiments, responsive to the controller 1104 determining that the temperature of the propagation fluid is greater than the second set point, the controller 1104 may be configured to stop power flowing from the power source(s) 1013, for example, by transmitting a signal to the power distribution circuit 1010, to stop power flowing to the heating element(s) causing them to turn off.

In some cases, the positioning of the heating element(s) 1102 within the apparatus 1000 can lead to a non-uniform distribution of heat throughout the propagation fluid. For example, the heating elements 1102 may heat a top level of the propagation fluid within the reservoir 204, causing a temperature differential between fluid closer to the heating elements 1102 and the fluid further from the heating elements 1102. In some embodiments, the heating control system 1012 further comprises a pump 1300 provided within the housing 1001 of the apparatus 1000. The pump 1300 may be a piston pump, for example. The pump 1300 is configured to cause movement or circulation of the propagation fluid within the reservoir 204 to thereby achieve a more uniform heat distribution and uniform temperature of the propagation fluid. By providing more propagation fluid with a more uniform temperature profile, improved impedance matching with fluid deployed on a contact surface of a target may be achieved, which may allow for improved images to be acquired.

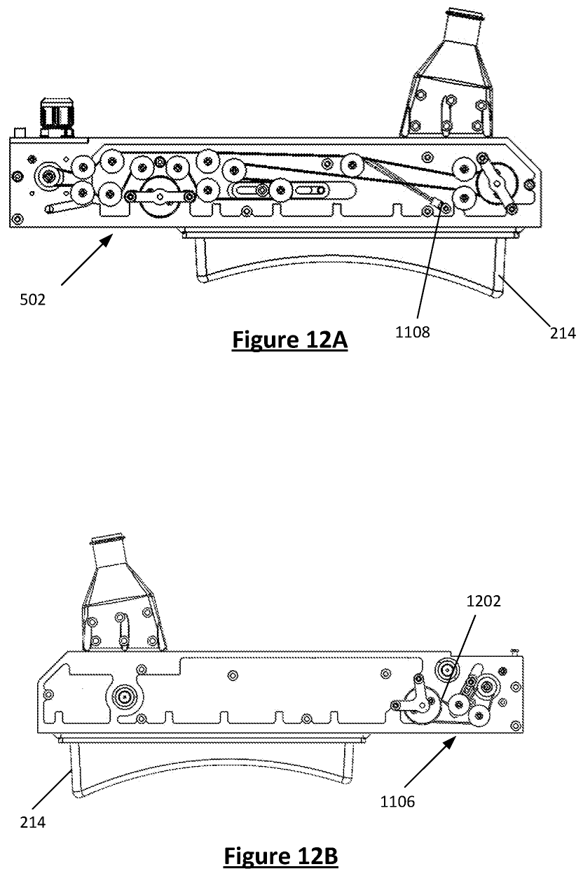

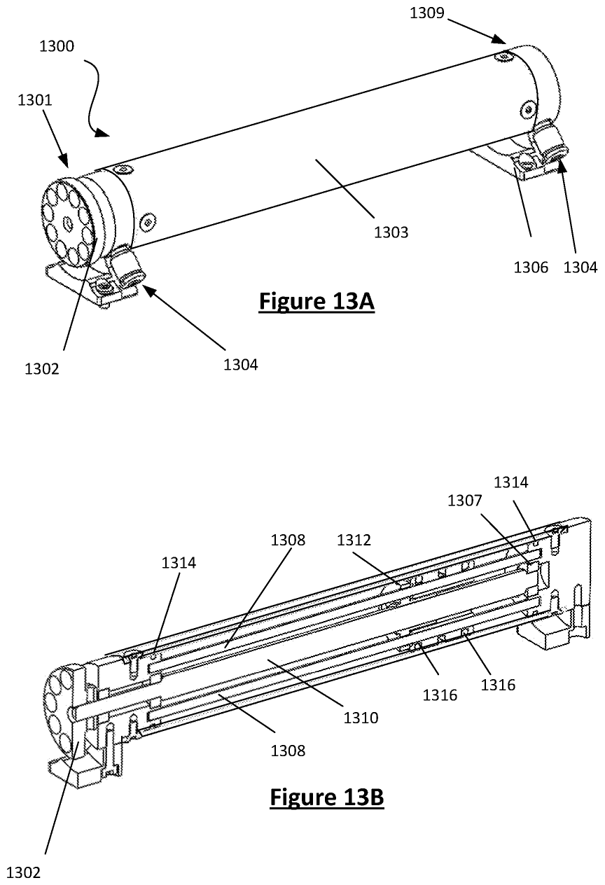

The apparatus 1000 may comprise one or more motor systems, such as transducer motor system 502 and/or pump motor system 1106. FIG. 12A is a first side sectional view of the apparatus 1000 and depicts the transducer motor system 502. Motor system 502 may operate to move a transducer mount within the housing 1001, as previously described. FIG. 12A also shows a temperature sensor 1108 provided towards the lower rear of apparatus 1000. The temperature sensor 1108 in FIG. 12A may be proximate to flange 214, thereby providing temperature readings from a region closer to the bottom of the reservoir 204. The placement of temperature sensor 1108 in FIG. 12A, being proximate to the bottom of the reservoir 204, allows for measurement of the propagation fluid temperatures that are relatively far away from the heat pads 1102. The depicted placement of temperature sensor 1108 allows for a temperature difference to be determined between propagation fluid at a close point to the heating pads 1102, and propagation fluid further away from the heating pads 1102.

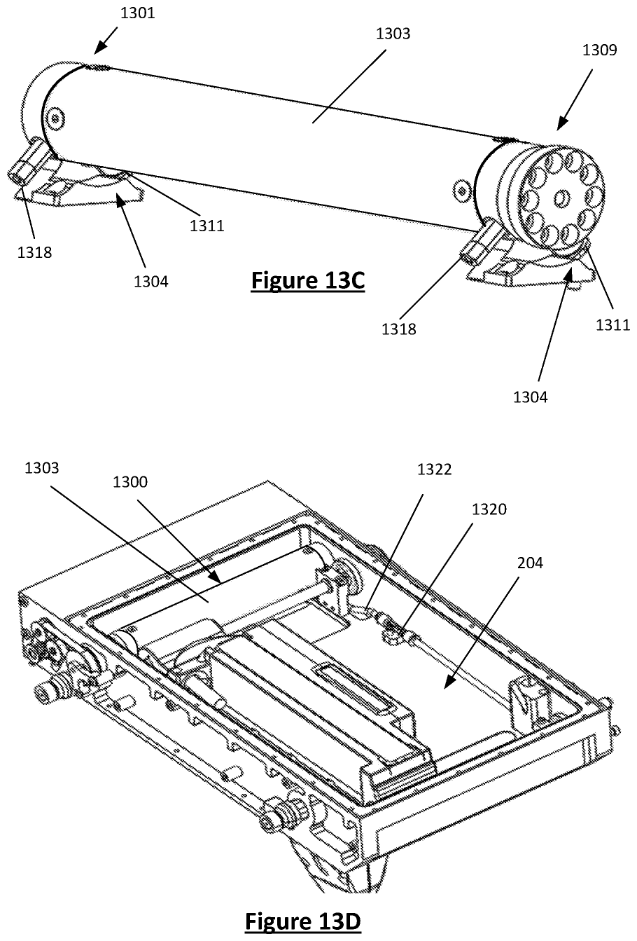

FIG. 12B is a second side sectional view of the apparatus 1000, showing the opposing side to FIG. 12A, and depicting the pump motor system 1106. Pump motor system 1106 may be configured to activate the pump 1300 within the housing 1001 of apparatus 1000, to cause propagation fluid within the reservoir 204 to circulate. Pump motor system 1106 may comprise a belt drive system 1202 that transfers torque from the motor 1107 (as also shown in FIG. 11B) to a magnetic coupler 1302 (FIGS. 13A and 13B). The magnetic coupler 1302 then transfers this torque to a second coupler 1307 connected or coupled to the pump 1300 which drives a screw nut 1312 back and forth, providing a piston pump action, as discussed below.

FIG. 13A is a perspective view of the pump 1300, according to some embodiments. Magnetic coupler 1302 may be coupled to a pump housing 1303 of the pump 1300. The magnetic coupler 1302 may be substantially similar to couplers 804, 806 as previously described.

Magnetic coupler 1302 may be installed on a section of the housing 1001 that abuts a wall of the reservoir 204, such that a matched magnetic coupling or second coupler 1307 on the motor system 1106 can drive the pump 1300, indirectly, or without direct contact. In some embodiments, pump 1300 is installed outside of, but in fluid communication with, the reservoir 204. In such embodiments, the magnetic coupler 1302 may be in contact with an interior compartment of housing 1001, such that a matched magnetic coupling (second coupler 1307) on the motor system 1106 can drive the pump 1300, indirectly, or without direct contact. Magnetic coupler 1302 may be positioned at or toward a first end of the pump housing 1303.

Pump 1300 may comprise the second coupler 1307. Second coupler 1307 may be installed opposed to the magnetic coupler 1302. Second coupler 1307 may be configured to engage a lead screw 1310 and allow the motor system 1106 to rotate the lead screw within pump housing 1303. Pump housing 1303 may comprise a hollow body having the first end 1301 and second end 1309. Second coupler 1307 may be connected to pump housing 1303 at or towards the second end 1309. Pump housing 1303 may comprise a substantially cylindrical body. Pump housing 1303 may be installed in fluid connection with reservoir 204, to mix propagation fluid stored therein. Pump housing 1303 may be installed within reservoir 204, to mix propagation fluid stored therein.

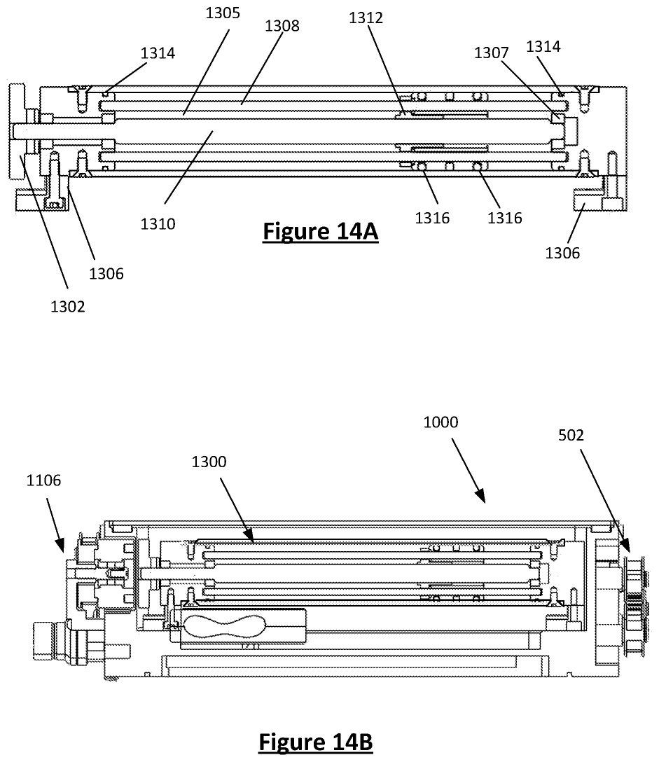

FIG. 13B, 13C and FIG. 14A depict sectional views of the pump 1300 of FIG. 13A, showing its internal components. Pump housing 1303 may define an internal pump reservoir 1305. The pump 1300 may comprise fluid port(s) 1304, 1311 (FIG. 13D) conveying propagation fluid to and from to the internal pump reservoir 1305. For example, and as illustrated, a first fluid port (e.g. outlet) 1304 may be disposed at or toward the first end 1301 of the pump 1300 and a second fluid port 1304 (e.g. outlet) may be disposed at or toward the second end 1309 of the pump 1300. The first and second fluid port 1304 may comprise a valve 1318, 1320, such as a check valve. The valve 1318 may be a one-way valve configured to allow flow in one direction, and restrict flow in an opposite direction. Similarly, the valves 1318, 1320 provided at the first and second fluid ports 1304 may be configured to allow fluid to be conveyed or expelled from the pump reservoir 1305 into the reservoir 204 but restrict or prevent fluid from being conveyed to the pump reservoir 1305 from the reservoir 204.

As illustrated in FIG. 13D, a conduit 1322, such as a tube, may extend from the fluid port(s) 1311 (e.g. inlet port) of the pump reservoir 1305 into the reservoir 204 such that the propagation fluid may be conveyed or draw from a location displaced or distanced from the pump housing 1303, and for example, the outlet port(s) 1304. As depicted in FIG. 13D, an inlet to the conduit may be located substantially mid-way along a length of the housing 1101 or reservoir 204. In some embodiments, a first inlet to the conduit 1322 is disposed toward or at a first longitudinal wall of the reservoir 204 and a second inlet to the conduit is disposed toward or at a second, and opposite longitudinal wall of the reservoir 204. The valve 1320, such as a check valve, or one-way valve, may be provided within the conduit 1322, for example, toward or at the inlet of the conduit. The valve 1320 may be configured to allow fluid to be conveyed or drawn into the pump reservoir 1305 (via the conduit) from the reservoir 204 but restrict or prevent fluid from being conveyed from the pump reservoir 1305 (and conduit) to the reservoir 204.

By drawing fluid from a location within the reservoir that is spaced apart from the outlet ports 1314, the pump 1300 allows for a relatively thorough mixing of the propagation fluid within the reservoir 204 to thereby better distribute heat, mixing cooler fluid with the warmer fluid (closest to the heating elements 1102) aiding in achieving a more consistent temperature throughout the fluid in the reservoir 204 of the housing 1001.

The pump housing 1303 may be supported by alignment rods 1308. Alignment rods 1308 may ensure stability of the pump 1300 during use, by resisting the rotation of a shaft or screw 1310, and ensuring linear motion of screw nut 1312. The pump 1300 may be affixed to the housing 1001 of apparatus 1000 by pump mount(s) 1306.

The pump 1300 may operate by the action of a screw nut 1312 moving along the shaft or screw 1310. As illustrated the shaft or screw 1310 extends between the first end of the pump housing 1303 and the second end of the pump housing 1303. The screw 1310 is connected to the magnetic coupler 1307 (e.g. magnetic shaft coupling). As the magnetic coupler 1307 is magnetically coupled to magnetic coupler 1302, as the magnetic coupler 1302 is rotated by the motor 1107, the magnetic coupler 1307, thereby causing rotation of the shaft or screw 1310. Screw 1310 may have a threaded surface, which, when rotated by the magnetic coupler 1307, causes matching threaded engagements on the screw nut 1312 to move the screw nut 1312 (backwards and forwards) along the length of the screw 1310, and thereby providing a piston pump action. Screw nut 1312 may further comprise one or more piston seals 1316. Piston seals 1316 may comprise O-rings, and be configured to engage an interior surface of the pump housing 1303, to prevent fluid transferring over the screw nut 1312 within the pump housing 1303. Pump housing 1303 may also comprise static seals 1314. Static seals 1304 may comprise O-rings.

In use, the movement of screw nut 1312 along screw 1310 simultaneously draws propagation fluid into the internal pump reservoir 1305 through the inlet fluid ports 1311 from the reservoir 204, and forces or expels propagation fluid from the internal pump reservoir 1305 through the outlet fluid port(s) 1304 into the reservoir 204. This process repeats in reverse as the screw nut 1312 travels in an opposing direction. When installed in fluid communication with reservoir 204, the pump 1300 provides a mixing action, mixing the propagation fluid, and allowing for a more uniformly distribution of the heating effect of the heating element(s) 1102.

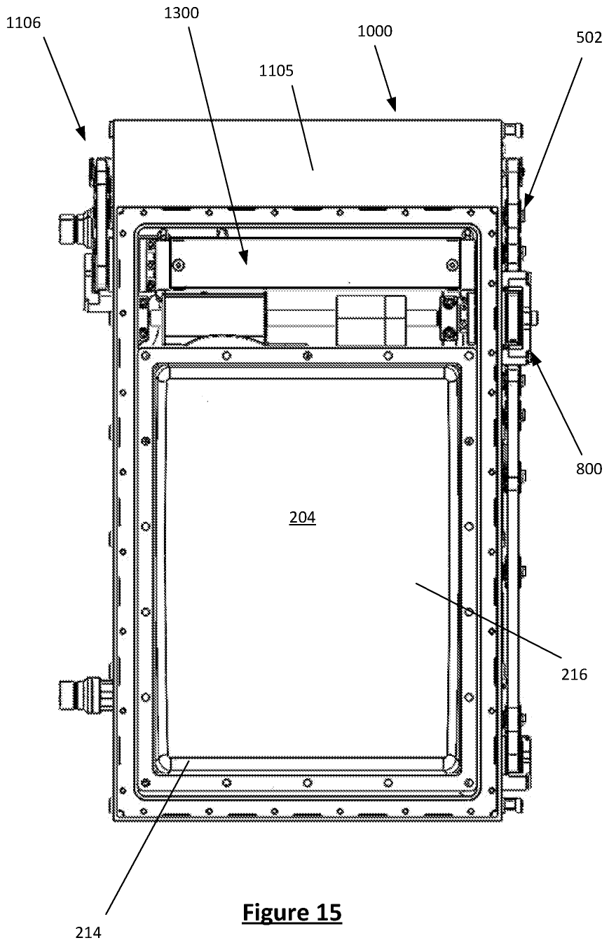

FIGS. 14B and 15 are sectional views of the apparatus 1000 depicting an installation location of pump 1300 within the housing 1001 of apparatus 1000. FIG. 14B depicts pump 1300 provided toward an end of the housing 1001. In some embodiments, the pump 1300 is disposed alongside or in close proximity to the electronics bay 1105. The pump 1300 may be disposed alongside or in close proximity to the driving mechanism 800. As illustrated in FIG. 15, the pump 1300 is disposed between the electronics bay 1105 and the driving mechanism 800.

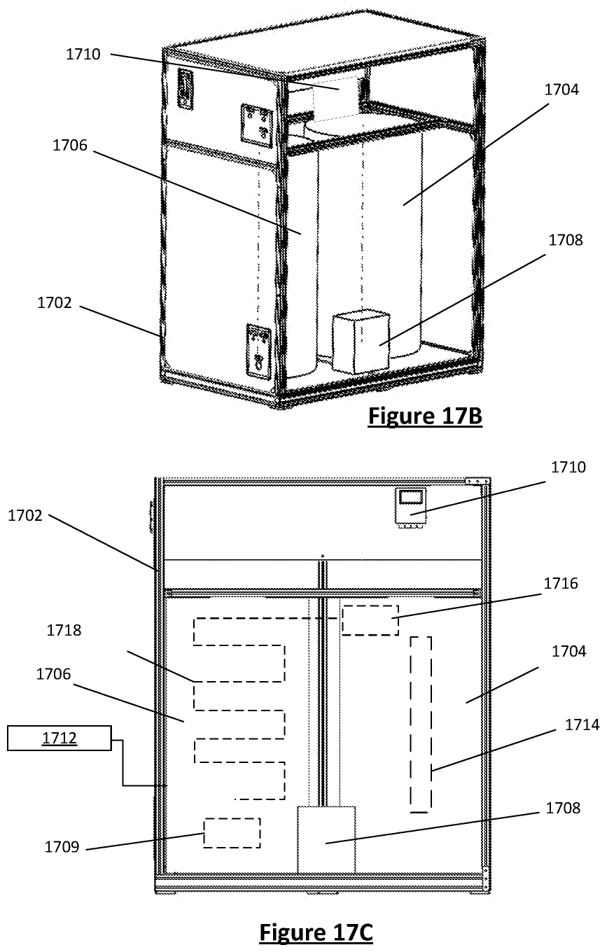

FIGS. 17A, 17B, and 17C depict a propagation fluid heater and storage tank 1700. The propagation fluid heater 1700 and storage tank may use a heat exchange mechanism to heat propagation fluid stored therein. Fluid heater and storage 1700 may comprise a housing 1702 defining or comprising a water tank 1704 and a propagation fluid tank 1706. The water tank is configured to receive and retain water, and the propagation fluid tank 1706 is configured to receive and retain propagation fluid. For example, the water tank 1704 and a propagation fluid tank 1706 may be deployed alongside each other. For example, a side wall of the water tank 1704 may be aligned with a respective side wall of the propagation fluid tank 1706. The walls of the two tanks 1704 may be flush with one another or gap may be provided therebetween. One or more heating elements 1714 may be provided within the water tank 1704 to allow for selective heating of water provided in the water tank 1704. For example, heating elements 1714 may be longitudinal and may extend from an upper location within the water tank 1704 to a lower location within the water tank 1704 to thereby more evenly heat water in the water tank 1704. A water pump 1716 is provided towards an upper end of the water tank 1704. The water pump 1716 is in fluid communication with the water in the water tank 1704 and is configured to selectively convey or pump water from the water tank 1704 through a conduit 1718, such as a coil extending from the water tank 1704 to the propagation fluid tank 1706. As illustrated, the conduit 1718 extends from toward an upper end of the propagation fluid tank 1706 toward a lower end of the propagation fluid tank 1706, and back to the water tank 1704. The water pump and conduit 1718 thereby provide a closed loop heating circuit conveying heat from the heated water in the water tank 1704 to the propagation fluid in the propagation fluid tank 1706. In other words, as heated water is pumped through the conduit 1718, heat is transferred to the propagation fluid within the propagation fluid tank. For example, the conduit 1718 may be arranged in a zig-zag configuration to thereby better transfer heat from the heated water passing through the conduit 1718 to the propagation fluid provided within the propagation fluid tank 1706. In some embodiments, the conduit 1718 comprises a coil configuration.

A fluid pump 1709 may be provided within the propagation fluid tank 1706 and may be configured to circulate the propagation fluid within the propagation fluid tank 1706, for example, conveying fluid from the bottom of the propagation fluid tank 1706 to the top, to thereby improve even mixing of the propagation fluid leading to a more consistent temperature of the propagation fluid within the propagation fluid tank 1706.

The propagation fluid heater and storage tank 1700 may comprise a fluid dispenser 1712 is fluid communication with the propagation fluid tank 1706 to allow fluid to be selectively dispensed from the propagation fluid tank 1706. The propagation fluid heater and storage tank 1700 comprises a fluid port (not shown) by which propagation fluid can be conveyed into the propagation fluid tank 1706 from an external reservoir (not shown) to replace propagation fluid being dispensed from the propagation fluid tank 1706 via dispenser 1712. A pump 1708, such as a diaphragm pump, may be provided at the fluid port (not shown) to maintain constant pressure in the propagation fluid tank 1706. The pump 1708 may be configured to actuate as propagation fluid is dispensed, to thereby draw or suck new propagation fluid from the external reservoir and push it, under pressure, into the propagation fluid tank 1706 to mix with propagation fluid already contained therein. The propagation fluid in the external reservoir may be at ambient temperature, and as it mixes with the propagation fluid in the propagation fluid tank and is subjected to heat from the conduit, its temperature increases.

The propagation fluid heater 1700 and storage tank may comprise a controller 1710 configured to control the temperature of the heating element(s) 1714, to activate the water pump 1716, and/or to activate the fluid pump 1709. The controller 1710 may be configured to receive user input to allow a user 104 to set a desired propagation fluid temperature. Controller 1710 be configured to selectively activate and deactivate the water pump 1716 and/or the fluid pump 1709 to control the circulation of the water and/or the propagation fluid, respectively.

By providing an indirect heating means as described, the heat of the propagation fluid is naturally self-limiting, and cannot be heated beyond the controlled temperature of the water. This is beneficial in embodiments where the propagation fluid is oil, which may pose an ignition risk in the event of an equipment failure. In the described arrangement, even in the event of a heating element 1714 failure, such as being stuck in an “on” position, the maximum temperature that oil could be heated to is 100° C.—which is well below the ignition temperature of vegetable or canola oil (being approximately 200° C.).

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. An apparatus for facilitating ultrasound scanning of objects, the apparatus comprising:
a housing defining a reservoir, the housing comprising:
a body comprising a transducer mounting, the transducer mounting configured to support an ultrasound transducer;
a flange extending from the body to define side walls of the reservoir; and
a first flexible membrane mounted on a free end of the flange, the first membrane defining an external contact surface for making contact with an object to be scanned, and the first flexible membrane configured to allow ultrasound waves to propagate therethrough;
wherein the reservoir is configured to retain ultrasound propagation fluid to facilitate ultrasound wave propagation between the transducer and the first flexible membrane.

2. The apparatus of claim 1, wherein the transducer mounting is disposed within the reservoir of the housing.

3. The apparatus of claim 1 or claim 2,
wherein the housing comprising a second membrane opposite to the first flexible membrane, and
wherein the transducer mounting is disposed on an external surface of the housing, such that ultrasound waves emitted from the transducer are configured to propagate through the second membrane into the reservoir, through ultrasound propagation fluid in the reservoir and through the first flexible membrane.

4. The apparatus of any one of the preceding claims, wherein the first flexible membrane comprises an elastomeric material.

5. The apparatus of any one of claims 2 to 4, wherein the membrane comprises a sheet of silicone having a thickness of about 0.1 mm to 3 mm.

6. The apparatus of any one of the preceding claims, wherein the free end of the flange comprises a substantially curved surface such that the first flexible member mounted thereon provides a substantially curved external contact surface.

7. The apparatus of any one of the preceding claims, wherein the flange comprises, or is composed of, moulded silicone.

8. The apparatus of any one of the preceding claims, wherein the flange and/or the first flexible membrane are removably coupled to the housing.

9. The apparatus of any one of the preceding claims, further comprising a track disposed along an inner wall of the housing, the track being configured to cooperate with the transducer mounting to allow the transducer mounting to be selectively moved along the track and assume a range of positions within the reservoir.

10. The apparatus of claim 9, wherein the track is configured to allow the mounting to move in a first direction and to move in a second direction, wherein the second direction is orthogonal to the first direction.

11. The apparatus of claim 9 or claim 10, wherein the transducer mounting comprises at least one shaft, and wherein the apparatus further comprises:
at least one motor, external to the reservoir;
at least one magnetic motor coupling connected to the motor and configured to magnetically engage at least one magnetic shaft coupling attached to the at least one shaft.

12. The apparatus of claim 11, wherein the at least one motor comprises at least one stepper motor.

13. The apparatus of claim 11, wherein the at least one motor comprises at least one servomotor.

14. The apparatus of any one of the preceding claims, wherein the housing comprises at least one handle extending from the housing to allow a user to hold the apparatus during operation.

15. The apparatus of any one of the preceding claims, wherein the reservoir is filled or substantially filled with ultrasound propagation fluid, and wherein the ultrasound propagation fluid is configured to propagate ultrasounds waves between the transducer and the first membrane.

16. The apparatus of any one of the preceding claims, further comprising: an ultrasound transducer mounted on the transducer mounting.

17. The apparatus of any one of the preceding claims, further comprising:
a pump provided within the housing, configured to mix ultrasound propagation fluid stored within the reservoir.

18. The apparatus of claim 17, wherein the pump further comprises a shaft connected to at least one magnetic shaft coupling and a nut, wherein the at least one magnetic shaft coupling is configured to magnetically couple to a magnetic motor coupling arranged to be driven by a motor, and responsive to rotation of the magnetic motor coupling, the magnetic shaft coupling rotates, thereby causing the shaft to rotate and the nut to move back and forth along the shaft providing a piston pump action.

19. The apparatus of any one of the preceding claims further comprising:
at least one heating element, provided within the housing, and configured to selectively heat ultrasound propagation fluid stored within the reservoir.

20. The apparatus of any one of the preceding claims, further comprising:
at least one temperature sensor, within the housing, configured to determine a temperature of ultrasound propagation fluid stored within the reservoir.

21. The apparatus of any one of claims 1 to 18, further comprising:
at least one heating element, provided within the housing, and configured to selectively heat ultrasound propagation fluid stored within the reservoir;
at least one temperature sensor, within the housing, configured to determine a temperature of ultrasound propagation fluid stored within the reservoir; and
a controller having a memory and one or more processors, wherein the controller is configured to:
receive temperature data from the at least one temperature sensor, the temperature data being indicative of the temperature of the propagation fluid within the reservoir;
responsive to the temperature of the propagation fluid being less than a first threshold level, activate the at least one heating element to thereby heat the propagation fluid; and
responsive to the temperature of the propagation fluid being greater than a second threshold level, deactivate the at least one heating element.

22. An apparatus comprising:
a body;
an ultrasound transducer connected to the body;
a flange extending from the body; and
a flexible membrane mounted on the flange,
wherein the flexible membrane, flange and body cooperate to define a reservoir between the ultrasound transducer and the flexible membrane, the reservoir being at least partially surrounded by the flange and configured to contain ultrasound propagation fluid between the ultrasound transducer and the flexible membrane.

23. A system comprising:
the apparatus of any one of the preceding claims, wherein the apparatus further comprises a winch mounting, and
a winch configured to couple to the winch mounting.

24. The system of claim 23, wherein the winch comprises a motorized winch.

25. The system of claim 23 or claim 24, wherein the system further comprises:
a controller having a memory and one or more processors,
wherein the memory comprises instructions for operating the winch according to one or more predefined settings; and
wherein the one or more processors are configured to execute the instructions to operate the winch.

26. The system of claim 25, wherein the controller comprises a user interface for receiving one or more values for the respective one or more predefined settings, and optionally operational zone ranges for the winch mounting.

27. A system comprising:
a first storage tank, configured to store a quantity of water;
a second storage tank, configured to store a quantity of ultrasound propagation fluid;
at least one heating element, provided within the first storage tank, and configured to selectively heat water stored within the first storage tank;
a conduit arranged to extend from a first fluid port in the first storage tank through the second storage tank and to a second fluid port in the first storage tank;
a pump, configured to convey water from the first storage tank through the conduit;
at least one temperature sensor, within the second storage tank, configured to determine a temperature of the ultrasound propagation fluid stored within the second storage tank; and
a controller, wherein the controller is configured to:
receive temperature data from the at least one temperature sensor, the temperature data being indicative of the temperature of the propagation fluid within the second storage tank;
operate the heating element to thereby heat water in the first storage tank;
responsive to the temperature of the propagation fluid within the second storage tank being less than a first threshold level, activate the pump to circulate the heated water from the first storage tank into the conduit in the second storage tank, thereby heating propagation fluid within the second storage tank; and
responsive to the temperature of the propagation fluid being greater than a second threshold level, deactivate the pump.

28. The system of claim 27, further comprising:
a propagation fluid pump provided within the propagation fluid tank and configured to circulate the propagation fluid within the propagation fluid tank.

29. The system of any one of claim 27 or 28, further comprising:
a dispenser configured to dispense ultrasound propagation fluid from the second storage tank.
